(19) United States
(12) Patent Application Publication
(10) Pub. No.: US 2013/0302861 A1
(43) Pub. Date: Nov. 14, 2013

(54) EXPRESSION CONSTRUCTS AND USES THEREOF IN THE PRODUCTION OF TERPENOIDS IN YEAST

(71) Applicant: Yissum Research Development Company of the Hebrew University of Jerusalem Ltd., (US)

(72) Inventors: Alexander VAINSTEIN, Rechovot (IL); Elena Marhevka, Rechovot (IL); Moran Farhi, Rehovot (IL); Hagai Abelovich, Rehovot (IL)

(73) Assignee: Yissum Research Development Company of the Hebrew University of Jerusalem Ltd., Jerusalem (IL)

(21) Appl. No.: 13/893,367

(22) Filed: May 14, 2013

Related U.S. Application Data
(60) Provisional application No. 61/646,397, filed on May 14, 2012.

(51) Int. Cl. C12P 5/00

(52) U.S. Cl. CPC C12P 5/007
USPC 435/131; 435/167; 536/23.2; 435/254.2; 435/166; 426/650; 426/531

(57) ABSTRACT

A method of producing at least one terpene in a yeast cell is disclosed. The method comprises exogenously expressing within the mitochondria of the yeast cell or directing localization thereto a terpene synthase.

### RELATED APPLICATION

This application claims the benefit of priority under 35 USC §119(e) of U.S. Provisional Patent Application No. 61/646,397 filed May 14, 2012, the contents of which are incorporated herein by reference in their entirety.

### SEQUENCE LISTING STATEMENT

The ASCII file, entitled 55903SequenceListing.txt, created on May 14, 2013, comprising 91,460 bytes, submitted concurrently with the filing of this application is incorporated herein by reference.

### FIELD AND BACKGROUND OF THE INVENTION

The present invention, in some embodiments thereof, relates to expression constructs and, more particularly, but not exclusively, to the uses thereof in the production of terpenoids in yeast.

Plants produce an extensive and diverse array of secondary metabolites which have been used by mankind for centuries. Common uses include pharmaceuticals, perfumes, coloring agents and food additives. Terpenoids (or isoprenoids) are an extremely diverse class of natural compounds with tens of thousands of identified structures which are used, or posse potential for use, in commercial applications. Common applications range from anti-cancer and anti-malarial drugs, insecticides, to coloring agents, flavors and fragrances. In many instances, however, even in native plants the levels of the compounds of interest are often too low to allow commercial exploitation. As full chemical synthesis of most terpenoids involves multiple steps and low yields current productions depends on either inefficient and expensive extraction methods, that utilize large amounts of intact native plants, or it's tissue culture, or semi-chemical synthesis, which relies on a biologically produced starting substrates. Novel approaches to this challenge include metabolic engineering of heterologous organisms, with the aim of achieving a protocol for the rapid and inexpensive high level production of plant terpenoids in organisms that are easily cultivated and extracted [Maury, J. et al., J. in Biotechnology for the Future (2005) 100: 19-51 (Springer-Verlag Berlin, Berlin)]. This type of so called green chemistry, or white biotechnology, harnesses the wealth of genetic information and advances in genetic engineering, bioinformatics, and systems biology to design specialized ‘cell factories’; these make advantage of biocatalized in-vivo processes forming a low energy consuming, low ecological impact, chiral specific and single entity synthesis procedures.

Despite the extreme structural diversity of terpenoids they are biosynthesized, in principle, form the same C₅ building blocks of isopentenyl diphosphate (IPP) and its isomer dimethylallyl diphosphate (DMAPP). Head-to-tail condensation of DMAPP with IPP units, catalyzed by isoprenyl pyrophosphate synthases, elongates the chain forming longer linear isoprenylpyrophosphates. Terpenoids are then produced by the action of terpene synthases (TPS) which are classified by the chain length they utilize as substrates: monoterpenes are produced from C₁₀ geranyl diphosphate (GDP), sesquiterpenes from C₁₅ farnesyl diphosphate (FDP), diterpenes from C₂₀ geranylgeranyl diphosphate (GGDP), etc. Two pathways have been identified as involved in isoprenoids production: the deoxyxylulose-5-phosphate (DXP) pathway, found in most prokaryotes and plant plastids, and the mevalonate pathway (MVA) found in higher eukaryotes and plant cytosol. The former includes seven reactions (catalyzed by seven enzymes) starting with pyruvate and glyceraldehyde 3-phosphate and the later pathway starts with the condensation of acetyl-CoA and includes five steps for the production of IPP. The MVA pathway is responsible for example for production of major components of biological membranes sterols and DXP pathway in plants generates carotenoid pigments.

Saccharomyces cerevisiae, also known as Baker's or Brewer's Yeast, has an extensive history of use in the area of food processing and is renowned as a biotechnological workhorse, alongside Escherichia coli. With its long history of industrial applications, this yeast has also been the subject of various studies in the principles of microbiology and extensive knowledge has accumulated about its physiology, biochemistry and genetics, furthermore numerous biochemical, genetic screening and molecular biology tools were developed. One of the major advantages of yeast over E. coli based platforms is that being a eukaryote, yeast can naturally support molecular/terpenoid backbone modification and functionalization by e.g. glycosylation, acetylation or cytochrome-P450 dependent oxygenation. Yeast are also known to produce ergosterols, the main fungal sterol, dolichols and ubiquinone via the cytosolic MVA pathway. Thus, yeast has been suggested as a platform for the heterologous production of terpenoids [Nevoigt, E., Microbiol. Mol. Biol. Rev. (2008) 72: 379-412; Kirby, J. and Keasling, J. D., Nat. Prod. Rep. (2008) 25: 656-661; Grabinskaa, K. and Palamarczyk, G., FEMS Yeast Research (2002) 2: 259-265].

Eukaryotes such as plants and yeasts utilize several subcellular organelles, the mitochondria, the endoplasmic reticulum and the peroxisome for distinct metabolic activities. This compartmentalization also reflects differences in metabolites' localization and distribution of concentrations in the cell. Metabolic pathway engineering can take advantages strategies that target enzymes of interest to specific cellular location, thereby exposing the enzyme to optimal substrate concentration and maximizing yields.

U.S. Pat. No. 8,062,878 relates to recombinant expression of terpenoid synthase enzymes [e.g. levopimaradiene synthase (LPS)] and geranylgeranyl diphosphate synthase (GGPPS) enzymes in cells (e.g. microbial cells, yeast cells, plant cells) for the production of diterpenoids (e.g. levopimaradiene).

U.S. Pat. No. 7,453,024 relates to genetic engineering of flavor, fragrance and bio-control agent development. Specifically, U.S. Pat. No. 7,453,024 provides isolated or recombinant nucleic acid or functional fragment thereof encoding a proteinaceous molecule essentially capable of isoprenoid bioactive compound (I.e. flavor, fragrance and/or bio-control agent) synthesis when provided with a suitable substrate under appropriate reaction conditions. For example, according to U.S. Pat. No. 7,453,024, the proteinaceous molecule is capable of synthesizing a monoterpene alcohol linalool when contacted with geranyl diphosphate (GPP) and/or a sesquiterpene alcohol nerolidol when contacted with farnesyl diphosphate (FPP) under appropriate reaction conditions.

U.S. Patent Application No. 20120107893 relates to the production of one or more terpenoids through microbial engineering, and relates to the manufacture of products comprising terpenoids by balancing between the upstream, IPP-forming pathway with the downstream terpenoid pathway of taxadiene synthesis. For example, U.S. 20120107893 relates to methods involving recombinantly expressing a taxadiene synthase enzyme and a geranylgeranyl diphosphate synthase (GGPPS) enzyme in a cell (e.g. bacterial cell, yeast cell) that overexpresses one or more components of the non-mevalonate (MEP) pathway.

PCT Publication No. WO 2011/074954 relates to a valencene synthase, to a nucleic acid encoding same, to a host cell (e.g. bacterial cell, yeast cell) comprising same and to a method for preparing valencene. According to WO 2011/074954, the method comprises converting farnesyl diphosphate to valencene in the presence of a valencene synthase.

Additional background art includes PCT Publication No. WO2012/156976.

### SUMMARY OF THE INVENTION

According to an aspect of some embodiments of the present invention there is provided a method of producing at least one terpene in a yeast cell, the method comprising exogenously expressing within the mitochondria of the yeast cell or directing localization thereto a terpene synthase, thereby producing the at least one terpene in the yeast cell.

According to an aspect of some embodiments of the present invention there is provided a method of producing at least one terpene in a yeast cell, the method comprising: (i) exogenously expressing within the mitochondria of the yeast cell or directing localization thereto a terpene synthase; (ii) exogenously expressing within the mitochondria of the yeast cell or directing localization thereto a farnesyl diphosphate synthase; and (iii) exogenously expressing within the yeast cell a mutated form of yeast 3-hydroxy-3-methylglutaryl-coenzyme A reductase (tHMG), thereby producing the at least one terpene in the yeast cell.

According to an aspect of some embodiments of the present invention there is provided a nucleic acid construct, comprising a nucleic acid sequence encoding an enzyme selected from the group consisting of a terpene synthase and an enzyme in a terpenoid/sterol pathway which catalyzes formation of a farnesyl diphosphate (FDP), the nucleic acid sequence further comprising at least one cis-acting regulatory element active in a yeast cell for directing expression of the enzyme in the yeast cell and a nucleic acid element for directing expression of the enzyme or localization thereof in the mitochondria of the yeast cell.

According to an aspect of some embodiments of the present invention there is provided a yeast cell comprising in a mitochondria thereof an exogenously expressed terpene synthase and/or an exogenously expressed enzyme in a terpenoid/sterol pathway which catalyzes formation of a farnesyl diphosphate (FDP).

According to an aspect of some embodiments of the present invention there is provided a method of producing terpene in a yeast cell, comprising: (a) generating and/or increasing content of at least one terpene in the yeast cell according to the method of some embodiments of the present invention; and (b) isolating the terpene from the yeast cell, thereby producing the terpene.

According to an aspect of some embodiments of the present invention there is provided a method of producing terpene in a yeast cell, comprising: (a) providing the yeast cell of some embodiments of the present invention, and (b) isolating the terpene from the yeast cell, thereby producing the terpene.

According to an aspect of some embodiments of the present invention there is provided an isolated terpene produced by the method of some embodiments of the present invention.

According to an aspect of some embodiments of the present invention there is provided a method of producing a commodity selected from the group consisting of a natural flavor, a food product, a food additive, a fragrance, a cosmetic, a later/rubber, a fuel, a pesticide and a therapeutic agent, comprising producing terpene according to the method of some embodiments of the present invention and incorporating the terpene in a process for manufacturing a commodity, thereby producing the commodity.

According to some embodiments of the invention, the terpene synthase is translationally fused to a mitochondrial localization signal (MLS) peptide.

According to some embodiments of the invention, the method further comprises exogenously expressing within the yeast cell an enzyme in a terpenoid/sterol pathway which catalyzes formation of a farnesyl diphosphate (FDP).

According to some embodiments of the invention, the exogenously expressing within the yeast cell the enzyme in the terpenoid/sterol pathway which catalyzes formation of the farnesyl diphosphate is effected in the mitochondria of the yeast cell or by directing localization of the enzyme to the mitochondria of the yeast cell.

According to some embodiments of the invention, the enzyme in the terpenoid/sterol pathway is translationally fused to a mitochondrial localization signal (MLS) peptide.

According to some embodiments of the invention, the method further comprises exogenously expressing within the yeast cell a mutated form of yeast 3-hydroxy-3-methylglutaryl-coenzyme A reductase (tHMG).

According to some embodiments of the invention, the method further comprises exogenously expressing within the yeast cell a terpene synthase, wherein the terpene synthase is not expressed in, or directed to the mitochondria.

According to some embodiments of the invention, the terpene synthase is selected from the group consisting of a valencene synthase, a linalool synthase, a phytoene synthase, an amorphadiene synthase, a limonene synthase and a taxadiene synthase.

According to some embodiments of the invention, the enzyme in the terpenoid/sterol pathway is selected from the group consisting of a geranyl diphosphate synthase, a farnesyl diphosphate synthase and a geranylgeranyl diphosphate synthase.

According to some embodiments of the invention, the at least one terpene is a plant terpene.

According to some embodiments of the invention, the at least one terpene is a sesquiterpene.

According to some embodiments of the invention, the at least one terpene is selected from the group consisting of a sesquiterpene, a hemiterpene, a monoterpene, a diterpene, a sesterterpene, a triterpene, a sesquarterpene, a tetraterpene and a polyterpene.

According to some embodiments of the invention, the at least one terpene is selected from the group consisting of a taxadiene, a linalool, a valencene, a phytoene, an amorpha-4,11-diene, a limonene and a farnesyl diphosphate.

According to some embodiments of the invention, the terpene synthase and/or the farnesyl diphosphate synthase is translationally fused to a mitochondrial localization signal (MLS) peptide.

According to some embodiments of the invention, the terpene synthase comprises an amorphadiene synthase.

According to some embodiments of the invention, the terpene synthase comprises a valencene synthase.

According to some embodiments of the invention, the method further comprises exogenously expressing within the yeast cell a terpene synthase, wherein the terpene synthase is not expressed in or directed to the mitochondria.

According to some embodiments of the invention, the nucleic acid element encodes a mitochondrial signal peptide fused in frame to the enzyme.

According to some embodiments of the invention, the nucleic acid construct further comprises a nucleic acid sequence encoding a mutated form of yeast 3-hydroxy-3-methylglutaryl-coenzyme A reductase (tHMG).

According to some embodiments of the invention, the yeast cell further comprises an exogenously expressed mutated form of yeast 3-hydroxy-3-methylglutaryl-coenzyme A reductase (tHMG).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are graphs depicting yeast expressing plant sesquiterpene synthases Cstps1 or ADS which produce valencene or amorphadiene. FIG. 1A illustrates the total ions GC-MS chromatograms of volatiles collected form yeast expressing Cstps1 compared to yeast carrying an empty vector (control); and FIG. 1B illustrate terpenoids production in yeast expressing ADS as compared to yeast carrying an empty vector (control). Compounds were identified by comparison of RT and MS to those obtained from authentic standard/A. annua extract and NIST library.

FIGS. 2A-B are graphs depicting sesquiterpenes valencene and amorpha-4,11-diene production levels by engineered S. cerevisiae which can be elevated by over-expression of tHMG and FDPS. FIG. 2A illustrates valencene production as measured from GC-MS analysis of yeast cultures from W3031A background expressing Cstps1 (strain M208), Cstps1 and tHMG (strain M287), Cstps1 and FDPS (strain M290) or Cstps1, tHMG and FDPS (strain M144); and FIG. 2B illustrates amorphadiene production levels, as measured by GC-MS analysis, by yeast from BDXe background transformed with ADS alone (strain M263) or with ADS, tHMG and FDPS together (strain M1057). All experiments were performed by growing cells for 6 days in a biphasic batch culture supplemented with CuSO₄. Data is reported as mean±S.E. from a minimum three replicates.

FIGS. 3A-C are graphs depicting that targeting of terpene synthases to the yeast mitochondria greatly improves production levels. Plant sesquiterpenes produced by yeast after 6 days of growth in a dodecane biphasic batch culture and as measured by GC-MS analysis. FIG. 3A illustrates the effect of targeting Cstps1 to the mitochondria of W3031A yeast strain transformed with pMY5-mtCstps1, tHMG and FDPS (strain M201) or with pMY5-mtCstps1, Cstps1, tHMG and FDPS (strain M202) as compared to yeast cultures transformed with the same gene constructs except pMY-mtCstps1 (strains M135 and M144); FIG. 3B illustrates the change in valencene production in the BDXe strain background when targeting Cstps1 to the mitochondria (strain M242) versus expressing the cytosolic Cstps1 (strain M212) with or without co-expression of tHMG (strains M243 and M241)); and FIG. 3C illustrates elevation of amorphadiene production in BDXe yeast strain expressing mtADS (strain M213), tHMG, FDPS and mtADS (strain M1058) as compared to a BDXe lines expressing ADS (strain M263) or ADS, tHMG and FDPS (strain M1057). Data is reported as mean±S.E. from a minimum three replicates.

FIG. 4 is a graph depicting that co-expression of mtFDPS and mtTPS enhances terpenoid production levels. Amorphadiene produced by metabolically engineered yeast after 6 days of growth in a dodecane biphasic batch culture and as measured by GC-MS analysis. BDXe yeast strain expressing tHMG was transformed with ADS and FDPS (strain M1057) or with mtADS and FDPS (strain M1058), or ADS and mtFDPS (strain M1059) or mtADS and mtFDPS (strain M246). Data is reported as mean±S.E. from a minimum three replicates.

FIG. 5 is a schematic illustration of a pMY5 yeast expression plasmid.

FIG. 6 is a schematic illustration of a pMY6 yeast expression plasmid.

FIG. 7 is a schematic illustration of a pMY6L yeast expression plasmid.

FIG. 8 is a schematic illustration of a pδE yeast integrating expression plasmid.

FIG. 9 is a schematic illustration of a pδ-tHMG vector.

FIG. 10 is a schematic illustration of a pδ-FDPS vector.

FIG. 11 is a schematic illustration of a pMY5-Cstps1 vector.

FIG. 12 is a schematic illustration of a pδE-Cstps1 vector.

FIG. 13 is a schematic illustration of a pMY5-ADS vector.

FIG. 14 is a schematic illustration of a pδE-ADS vector.

FIG. 15 is a schematic illustration of a pMY5-mtCstps1 vector.

FIG. 16 is a schematic illustration of a pδE-mtCstps1 vector.

FIG. 17 is a schematic illustration of a pδE-mtADS vector.

FIG. 18 is a schematic illustration of a pδ-mtFDPS vector.

FIG. 19 is a schematic illustration of the mevalonic acid (MVA) pathway in S. cerevisiae. Genes that were integrated into the pathway (underlined) and those that were deleted (underlined and marked with D) are indicated. tHMG—truncated 3-hydroxy-3-methylglutarylcoenzyme A reductase, FDPS—heterologous farnesyl diphosphate synthase, CsTPS1—valencene synthase, and AaADS—amorpha-4,11-diene synthase; mt denotes mitochondrion-targeting sequence fused to the corresponding gene.

FIG. 20 is a schematic illustration of the mevalonic acid (MVA) and non-mevalonate (MEP) pathways, illustrated are various terpenoids produced from isoprenoids by different classes of terpene synthases.

### DESCRIPTION OF SPECIFIC EMBODIMENTS OF THE INVENTION

The present invention, in some embodiments thereof, relates to expression constructs and, more particularly, but not exclusively, to the uses thereof in the production of terpenoids in yeast.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The biologically and commercially important terpenoids are a large and diverse class of natural products which posses various uses ranging from therapeutics (e.g. anti-cancer and anti-malarial drugs), insecticides, to coloring agents, flavors and fragrances. However, in many instances, even in native plants, the levels of these compounds are often too low to allow commercial exploitation and are thus targets of metabolic engineering. Yet, in the context of metabolic engineering, the otherwise well-documented spatial subcellular arrangement of metabolic enzyme complexes has been largely overlooked.

While reducing the present invention to practice, the present inventors have surprisingly uncovered that yeast comprise a farnesyl diphosphate (FDP) pool within its mitochondria, which is available for the synthesis of terpenes. This is especially surprising since none of the key genes involved in terpene synthesis is expressed by the mitochondrial genome or localized to the mitochondria (see e.g. Saccharomyces Genome Database (SGD) at http://www(dot)yeastgenome(dot)org, The Yeast Resource Center (YRC) at http://depts(dot)washington(dot)edu/yeastrc, and Organelle DB at http://organelledb(dot)lsi(dot)umich(dot)edu). The present inventors have developed transgenic yeast for efficient production of terpenoids utilizing this FDP pool and by increasing the existing FDP pool. This system was harnessed towards generating terpenes of interest by expressing mitochondria targeted terpene synthases (TPSs) and optionally increasing the terpene production level by expressing a mitochondrial targeted farnesyl diphosphate synthase (FDPS) in yeast cells. The present inventors were able to increase terpene synthesis is yeast to an unprecedented level which is of industrial value.

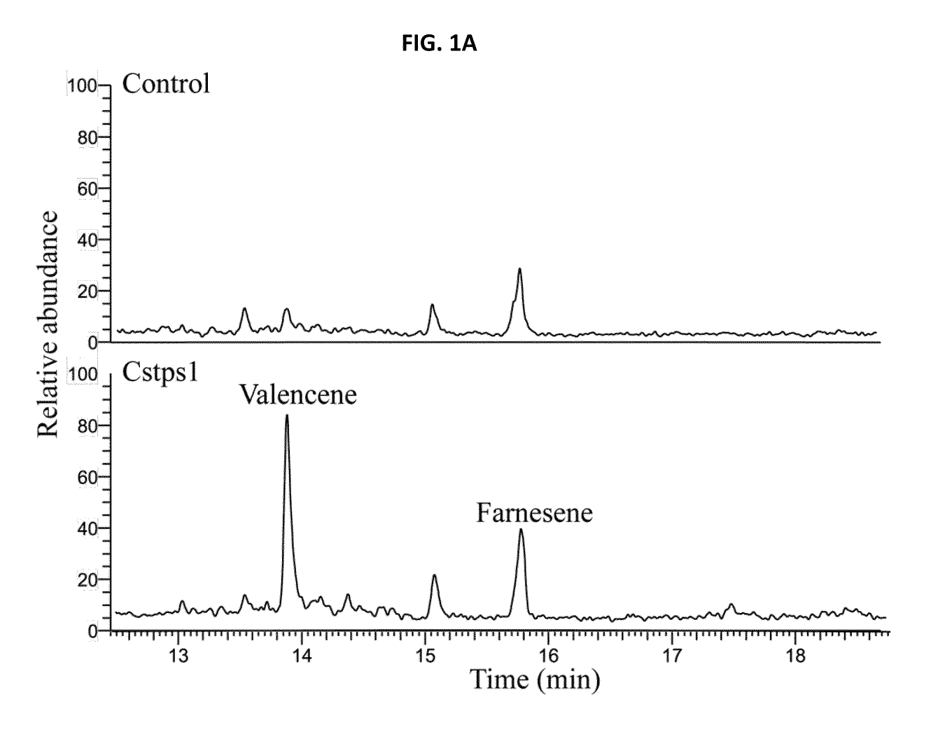

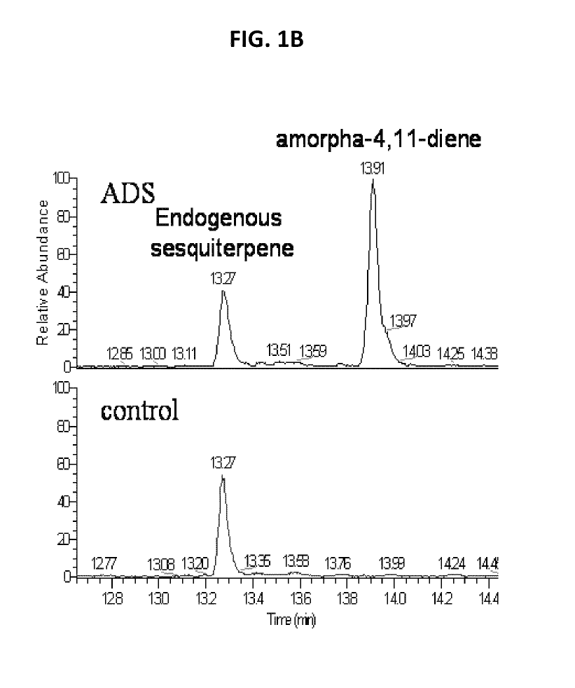

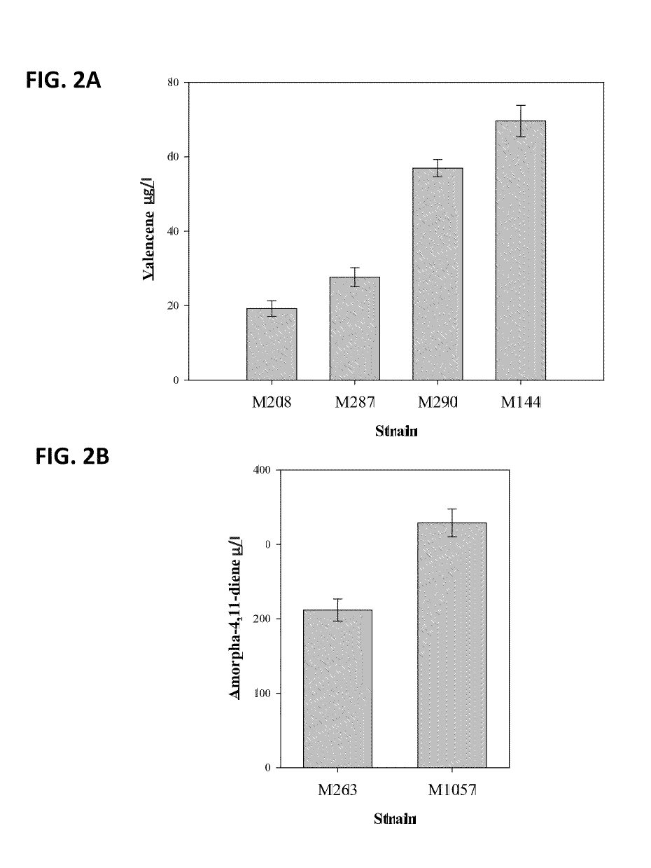

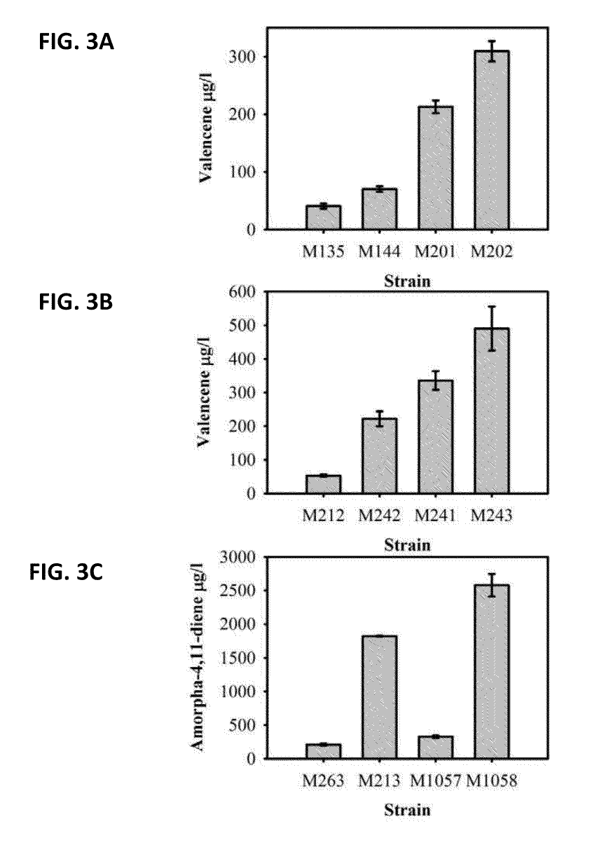

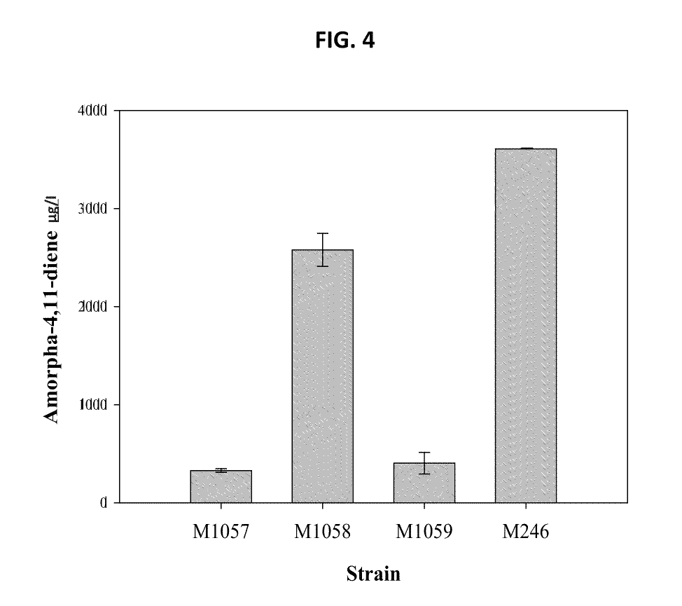

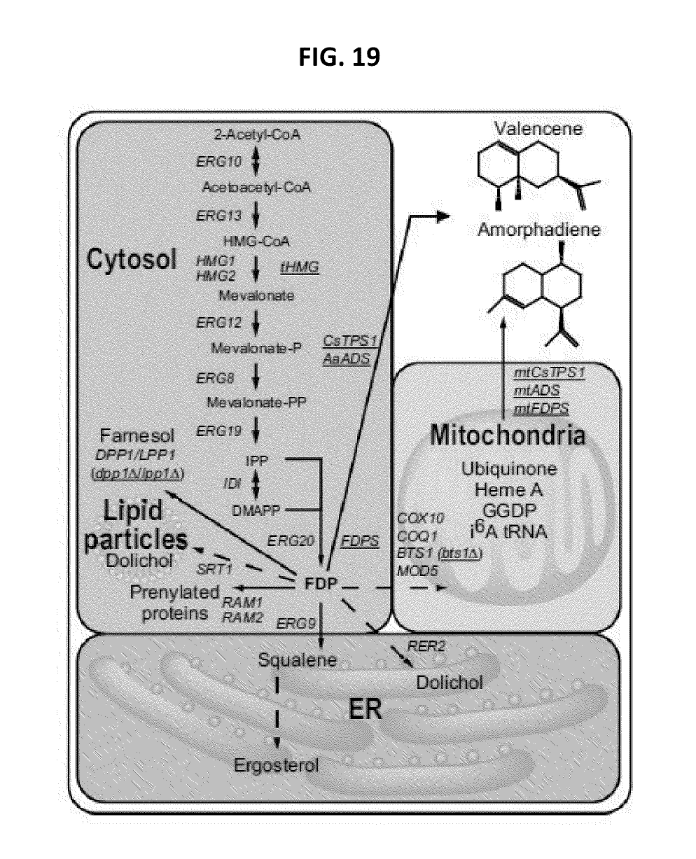

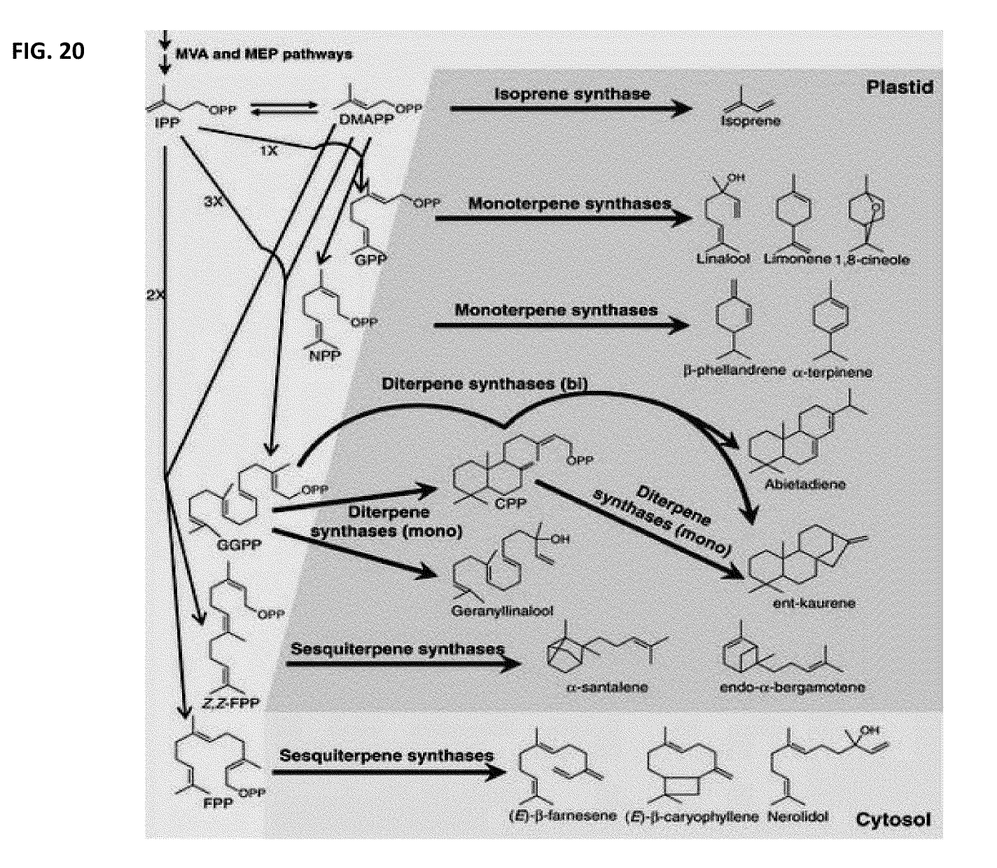

As is shown hereinbelow and in the Examples section which follows, the present inventors have enhanced production of plant sesquiterpenes in yeast by increasing flux in the mevalonic acid (MVA) pathway toward FDP (see schematic illustration in FIG. 19). Initially, production of plant terpenoids was illustrated upon expression of plant sesquiterpene synthases in yeast (FIGS. 1A-B). Next, production levels of valencene and amorphadiene were shown to be elevated by overexpressing in yeast a genome integrated form of an N′ terminal truncated hydroxymethylglutaryl-CoA (HMG-CoA) reductase (tHMG) and/or FDPS (FIGS. 2A-B and FIGS. 3A-C). Importantly, harnessing a different cellular compartment, namely the mitochondria, for a plants' FDPS and TPSs further elevated levels of sesquiterpene of interest. Specifically, an enhancement of 8- and 20-fold in the production of valencene and amorphadiene was achieved, respectively, in yeast co-engineered with a truncated and deregulated HMG1, mitochondrion-targeted heterologous FDP synthase and a mitochondrion-targeted sesquiterpene synthase, i.e. valencene or amorphadiene synthase (FIGS. 3A-C and FIG. 4). The aforementioned validates beyond any doubt the value of the present methods in producing terpenoids in yeast.

Thus, according to one aspect of the present invention there is provided a method of producing at least one terpene in a yeast cell, the method comprising exogenously expressing within the mitochondria of the yeast cell or directing localization thereto a terpene synthase, thereby producing the at least one terpene in the yeast cell.

As used herein, the term “yeast cell” refers to an isolated cell or a cell culture of yeast cells. The yeast cell of the present invention may refer to a native (naturally occurring) yeast cell, yeast cell lines and genetically modified yeast cells (e.g., genetically modified to express genes which are not necessarily associated with terpene synthesis). According to the present invention, yeast cells are engineered to produce a terpene from an isoprene [e.g. farnesyl diphosphate (FDP)]. The yeast cells of the present invention may naturally produce terpenes or may not be natural terpene producers.

Any host yeast may be employed for the purposes of the present invention. Candidate yeasts can be selected on various relevant criteria before, during, or after attempting to engineer in a terpene synthase. These secondary criteria include glycolytic rates, specific growth rates, thermotolerance, overall process robustness, and so on. These criteria can be evaluated in host cells, engineered cells, cells that have been evolved, cells that have been subjected to mutagenesis and selection, or cells that have otherwise been modified and screened.

In some embodiments, the yeast is selected from the genera Saccharomyces, Candida, Pichia, Kluyveromyces, Issatchenkia, Yarrowia, Rhodotorula, Hansenula, Schizochytrium, or Thraustochytrium. Some exemplary yeast species include Saccharomyces cerevisiae, Hansenula ofunaensis, H. polymorphs, H. anomala, Schizochytrium limacinum, Issatchenkia orientalis, Thraustochytrium striatum, T. roseum, T. aureum, Candida sonorensis, Kluyveromyces marxianus, K. lactis, and K. thermotolerans.

According to one embodiment, the yeast is Saccharomyces cerevisiae. According to an embodiment, suitable strains of Saccharomyces cerevisiae include W3031A (MATa, ade2-1, trp1-1, leu2-3,112 his3-11,15 ura3-1) and BDXe (developed by the present inventors as a derivative of a commercial strain, generated following screening for uracil auxotrophy by selection on 5-FOA, as described in detail in the Examples section which follows).

As mentioned, the yeast cell is genetically modified to express in the mitochondria thereof an exogenous gene that enables production of a terpene. The term “exogenous” as used herein refers to genetic material (e.g., a gene, promoter or terminator) that is not native to the host strain. The term “native” is used herein with respect to genetic materials that are found (apart from individual-to-individual mutations which do not affect function) within the genome of wild-type cells of the host cell (non-genetically modified).

As used herein the term “terpene”, also refers to as terpenoid or isoprenoid, refers to an organic chemical derived from a five-carbon isoprene unit. Several non-limiting examples of terpenoids, classified based on the number of isoprene units that they contain, include: hemiterpenoids or hemiterpenes (1 isoprene unit); monoterpenoids or monoterpenes (2 isoprene units); sesquiterpenoids or sesquiterpenes (3 isoprene units); diterpenoids or diterpenes (4 isoprene units); sesterterpenoids or sesterterpenes (5 isoprene units); triterpenoids or triterpenes (6 isoprene units); sesquarterpenoids or sesquarterpenes (7 isoprene units); tetraterpenoids or tetraterpenes (8 isoprene units); and polyterpenoids or polyterpenes with a larger number of isoprene units (i.e. long chains of many isoprene units).

According to one embodiment, the terpene is a sesquarterpene.

According to one embodiment, the terpene is a plant terpene.

Exemplary terpenoids which may be produced according to the present invention include, but are not limited to, Amorpha-4,11-diene, Carotene, Cafestol, Camphor, Cembrene, Cineol, Citral, Citronellol, Cubebol, Eleutherobin, Farnesyl diphosphate, Farnesenes, Farnesol, Ferrugicadiol Geraniol, Geranylfarnesol, Ginkgolides, Humulene, Isopemaradiene, Isovaleric acid, Kahweol, Labdenediol, Levopimaradiene, Limonene, Linalool, Lycopene, Menthol, Nootkatone, Pinene, Prenol, Phytol, Phytoene, Pseudopterosins, Rebaudioside A, Sarcodictyin, Sandracopimaradiene, Sclareol, Squalene, Stevioside, Taxadiene, Terpineol, Tetraprenylcurcumene, Valencene, gamma-carotene, alpha-carotene and beta-carotene. Additional terpenes which may be produced by the present invention are listed hereinunder.

As used herein the phrase “producing at least one terpene” refers to at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold increase in the content of a terpene in the yeast cell as compared to a native yeast cell (i.e., a cell not modified with the polynucleotides of the invention, e.g., a non-transformed yeast cell of the same species) which is grown (or cultured) under the same (e.g., identical) growth conditions.

According to one embodiment, producing at least one terpene in a yeast cell refers to upregulating the biosynthesis of one terpene, two terpenes, three terpenes, four terpenes, five terpenes or more, such as the complete repertoire of terpenes which are associated with the upregulated pathway in a yeast cell.

According to alternative embodiments of the invention, producing a terpene refers to producing a terpene within a cell (e.g. yeast cell) which does not produce a terpene when non-transformed to express the exogenous polynucleotide of some embodiments of the invention.

According to a specific embodiment, the production of terpene is achieved by exogenously expressing within the mitochondria of the yeast or directing localization thereto of a terpene synthase.

As used herein the phrase “terpene synthase or TPS” refers to a polypeptide which catalyzes formation of a terpene from any TPS substrate e.g. farnesyl diphosphate (FDP), geranyl diphosphate (GDP), geranylgeranyl diphosphate (GGDP) or copalyl diphospate (CDP).

Thus, according to the present invention, any terpene synthase may be used to produce at least one terpene in a yeast cell.

Exemplary terpene synthases include, but are not limited to, sesquiterpene synthases (e.g. EC 4.2.3.22, EC 4.2.3.23 and EC 4.2.3.46) and monoterpene synthases (e.g. EC 3.1.7.11), amorphadiene synthase (e.g. EC EC 4.2.3.24), copalyl diphosphate synthase (kaurene synthase A) (e.g. EC 5.5.1.12), ent-kaurene synthase B (e.g. EC 4.2.3.19), farnesene synthase (e.g. EC 4.2.3.47), linalool synthase (e.g. EC 4.2.3.25), limonene synthase (e.g. EC 4.2.3.16), myrcene synthase (e.g. EC 4.2.3.15), phytoene synthase (e.g. EC 2.5.1.32), pinene synthase (e.g. EC 4.2.3.14), taxadiene synthase (e.g. EC 4.2.3.17), valencene synthase (e.g. EC 4.2.3.73), and vetispiridiene synthase.

According to one embodiment, the terpene synthase comprises amorphadiene synthase (ADS).

As used herein the phrase “amorphadiene synthase (ADS)” refers to a polypeptide which catalyzes formation of amorpha-4,11-diene from farnesyl diphosphate (FDP), essentially as shown in FIG. 19 and described in Example 1 of the Examples section which follows (e.g., EC 4.2.3.24).

Non-limiting examples of coding sequences of amorphadiene synthase catalytic activity are provided in GenBank Accession NOs. ADU25497.1 (SEQ ID NO: 31 for polypeptide) and GenBank Accession NO. HQ315833.1 (SEQ ID NO: 30 for polynucleotide) from Artemisia annua; GenBank Accession NOs. AEQ63683.1 (SEQ ID NO: 35 for polypeptide) and JF951730.1 (SEQ ID NO: 34 for polynucleotide) from a synthetic construct; and GenBank Accession NOs. AFA34434.1 (SEQ ID NO: 37 for polypeptide) and JQ319661.1 (SEQ ID NO: 36 for polynucleotide).

According to some embodiments of the invention, the polynucleotide comprises a nucleic acid sequence encoding a polypeptide having at least 80%, at least 81%, 82%, 83%, 84%, 85%, 86%, 87%, 88%, 89%, 90%, 91%, 92%, 93%, 94%, 95%, 96%, 97%, 98%, 99%, e.g., 100% sequence homology or identity to the polypeptide set forth in SEQ ID NO: 31 (GenBank Access No. ADU25497.1), wherein the polypeptide catalyzes the formation of amorpha-4,11-diene from farnesyl diphosphate (FDP).

Homology (e.g., percent homology, identity+similarity) can be determined using any homology comparison software, including for example, the BlastP or TBLASTN software of the National Center of Biotechnology Information (NCBI) such as by using default parameters, when starting from a polypeptide sequence; or the tBLASTX algorithm (available via the NCBI) such as by using default parameters, which compares the six-frame conceptual translation products of a nucleotide query sequence (both strands) against a protein sequence database.

For example, default parameters for tBLASTX include: Max target sequences: 100; Expected threshold: 10; Word size: 3; Max matches in a query range: 0; Scoring parameters: Matrix—BLOSUM62; filters and masking: Filter—low complexity regions.

According to some embodiments of the invention, the polynucleotide comprises a nucleic acid sequence having at least 80%, at least 81%, 82%, 83%, 84%, 85%, 86%, 87%, 88%, 89%, 90%, 91%, 92%, 93%, 94%, 95%, 96%, 97%, 98%, 99%, e.g., 100% sequence identity to the polynucleotide set forth in SEQ ID NO: 30 (GenBank Accession No. HQ315833.1), wherein the polynucleotide encodes a polypeptide which catalyzes the formation of amorpha-4,11-diene from farnesyl diphosphate (FDP).

According to one embodiment, the terpene synthase comprises valencene synthase.

As used herein the phrase “valencene synthase (Cstps1)” refers to a polypeptide which catalyzes formation of valencene from farnesyl diphosphate (FDP), essentially as shown in FIG. 19 and described in Example 1 of the Examples section which follows (e.g., EC 4.2.3.73).

Non-limiting examples of coding sequences of valencene synthase catalytic activity are provided in GenBank Accession NOs. AF441124 (SEQ ID NO: 28) for polynucleotide and AAQ04608 (SEQ ID NO: 29) for polypeptide—from Citrus sinensis. As well as GenBank Accession NOs. AAS66358 and AAX16077.

According to some embodiments of the invention, the polynucleotide comprises a nucleic acid sequence encoding a polypeptide having at least 80%, at least 81%, 82%, 83%, 84%, 85%, 86%, 87%, 88%, 89%, 90%, 91%, 92%, 93%, 94%, 95%, 96%, 97%, 98%, 99%, e.g., 100% sequence homology or identity to the polypeptide set forth in SEQ ID NO: 29 (GenBank Access No. AAQ04608), wherein the polypeptide catalyzes the formation of valencene from farnesyl diphosphate (FDP).

According to some embodiments of the invention, the polynucleotide comprises a nucleic acid sequence having at least 80%, at least 81%, 82%, 83%, 84%, 85%, 86%, 87%, 88%, 89%, 90%, 91%, 92%, 93%, 94%, 95%, 96%, 97%, 98%, 99%, e.g., 100% sequence identity to the polynucleotide set forth in SEQ ID NO: 28 (GenBank Accession No. AF441124), wherein the polynucleotide encodes a polypeptide which catalyzes the formation of valencene from farnesyl diphosphate (FDP).

According to some embodiments of the invention the nucleic acid sequence of each of the enzymes expressed according to the teachings of the invention may (or may not) further comprise a nucleic acid sequence encoding a mitochondrial localization signal (MLS) peptide to thereby direct localization of the polypeptide into the mitochondria of the cell. The MLS is cloned in frame to the coding sequence encoding the enzyme to ensure proper translation of the full-length protein and localization thereof to the mitochondria. The enzyme may be cloned N terminally or C-terminally of the enzyme such that a fusion protein (chimeric protein) is formed.

As used herein, the term “mitochondrial localization signal or MLS” refers to a short target peptide chain (e.g. about 10-60 amino acids long) that directs the transport of a protein (e.g. enzyme) to a mitochondria of a cell.

According to one embodiment, the MLS is cleaved from the protein (e.g. enzyme) by signal peptidases after the protein is transported to the mitochondria.

Non-limiting examples of mitochondrial signal peptides which can be conjugated to the nucleic acid sequence of some embodiments of the invention (e.g., by recombinant techniques) may be obtained from the following proteins: Nicotiana plubaginifolia atp2-1 gene for mitochondrial ATP synthase: GenBank Access Nos. CAA26620.1/X02868.1 (SEQ ID NOs: 38 and 39), Mitochondrial import receptor subunit TOM20: GenBank Access Nos. NP_{—}198909.1/NM_{—}123458.4 (SEQ ID NOs: 41 and 40), Arabidopsis thaliana 2-oxoglutarate dehydrogenase subunit E1: GenBank Access Nos. BAE99494.1/AK227494.1 (SEQ ID NOs: 42 and 43).

Non-limiting examples of mitochondrial signal peptides which can be conjugated to the nucleic acid sequence of some embodiments of the invention (e.g., by recombinant techniques) include: Saccharomyces cerevisiae COX4 mitochondrial targeting sequence (SEQ ID NO: 45 for the polypeptide; and SEQ ID NO: 44 for the polynucleotide; GenBank Access Nos. NP_{—}011328 and NM_{—}001181052, respectively), HSP60/YLR259c of Saccharomyces cerevisiae (SEQ ID NO: 47 for the polypeptide; and SEQ ID NO: 46 for the polynucleotide; GenBank Access Nos. NP_{—}013360 and NM_{—}001182146, respectively), SSC1/YJR045c of Saccharomyces cerevisiae (SEQ ID NO: 49 for the polypeptide; and SEQ ID NO: 48 for the polynucleotide; M27229 and AAA63792, respectively), CYB2/YML054C of Saccharomyces cerevisiae (SEQ ID NO: 51 for the polypeptide; and SEQ ID NO: 50 for the polynucleotide; CAA86721 and NM_{—}001182412, respectively) or may be obtained from the polypeptide subunit 9 of the FO ATPase of Neurospora crassa (SEQ ID NO: 25 for the polypeptide; and SEQ ID NO: 52 for the polynucleotide; NCU16027 and AGG16016, respectively).

According to some embodiments of the invention the sequence encoding the mitochondria signal peptide which is conjugated to nucleic acid sequence of some embodiments of the invention (e.g., to the nucleic acid sequence encoding a terpene synthase) is the Saccharomyces cerevisiae COX4 mitochondrial targeting sequence (SEQ ID NO: 45 for the polypeptide; and SEQ ID NO: 44 for the polynucleotide).

According to some embodiments of the invention the nucleic acid sequence encoding the terpene synthase further comprises a nucleic acid sequence encoding a mitochondrial signal peptide to thereby direct localization of the terpene synthase into the mitochondria of the cell.

Thus, as shown in FIGS. 3A-C and 4 and described in Examples 3 and 4 of the Examples section which follows, using a construct which includes the mitochondria signal peptide conjugated to terpene synthase (e.g. ADS, Cstps1) resulted in significantly higher levels of terpenes in the engineered yeast.

According to some embodiments of the invention the polypeptide (e.g. terpene synthase) is expressed within the mitochondria of the yeast cell.

DNA can be delivered into yeast mitochondria by microprojectile bombardment i.e. a method by which DNA coated on microbeads composed of tungsten or gold is introduced into living cells at high speeds. This technique is often referred to as biolistic (biological ballistic) transformation. The DNA delivered into mitochondria is subsequently incorporated into the mitochondrial DNA (mtDNA) by the highly active homologous recombination machinery operating in the yeast organelle. This strategy inserting exogenous genes into mtDNA and provide a powerful in vivo tool for the study mitochondrial biogenesis in yeast' Bonnefoy, N. & Fox, T. D. Directed alteration of Saccharomyces cerevisiae mitochondrial DNA by biolistic transformation and homologous recombination. Methods Mol Biol 372, 153-66 (2007) (hereby incorporated by reference in its entirety).

Importantly, the yeast may be transformed with one or more exogenous genes (as described above) and combinations may be achieved by crossing the different engineered yeast. Thus, genetic crosses may be used to generate yeast strains expressing combination of genes, for instance, two, three or more exogeneous genes.

For expression in mitochondria, the nucleic acid constructs may further include a mitochondrially active promoter.

In order to increase terpene synthase expression, the present inventors have also expressed (in addition to the mitochondria) a terpene synthase wherein the terpene synthase is not expressed in, or directed to the mitochondria. Thus, for example, a terpene synthase may further (in addition to the mitochondria) be expressed in a cytosol of a yeast cell.

According to some embodiments of the invention, the method further comprises exogenously expressing within the yeast cell an enzyme in a terpenoid/sterol pathway which catalyzes formation of a farnesyl diphosphate (FDP).

As used herein, the term “an enzyme in a terpenoid/sterol pathway” refers to a polypeptide which catalyzes directly or indirectly formation of a farnesyl diphosphate (FDP) in the terpenoid metabolic pathway.

As used herein the phrase “farnesyl diphosphate or FDP”, also referred to as Farnesyl pyrophosphate (FPP), is an intermediate in the HMG-CoA reductase pathway used in the biosynthesis of terpenes, terpenoids, and sterols.

Exemplary enzymes in the terpenoid/sterol pathway include, but are not limited to, geranyl diphosphate synthase (e.g. EC 2.5.1.29), farnesyl diphosphate synthase (e.g. EC 2.5.1.10), geranylgeranyl diphosphate synthase (e.g. EC 2.5.1.29), squalene synthase (e.g. EC 2.5.1.21), IPP isomerase (e.g. EC 5.3.3.2) and neryl diphosphate synthase (e.g. EC 2.5.1.28).

According to one embodiment, an enzyme in the terpenoid/sterol pathway comprises a Farnesyl diphosphate synthase (FDPS) e.g., EC 2.5.1.10.

Non-limiting examples of coding sequences of FDPS are provided in GenBank Accession NOs. NM_{—}124151 (SEQ ID NO: 26) for polynucleotide and NP_{—}199588 (SEQ ID NO: 27) for polypeptide—from Arabidopsis thaliana; as well as GenBank Accession NOs. NM_{—}202836 and NP_{—}974565 (for polynucleotide and polypeptide, respectively) from Arabidopsis; GenBank Accession NOs. NM_{—}002004 and NP_{—}001995 (for polynucleotide and polypeptide, respectively) from Homo sapiens; GenBank Accession NOs. XM_{—}707792 and XP_{—}712885 (for polynucleotide and polypeptide, respectively) from Candida albicans; and GenBank Accession NOs. XM_{—}422855 and XP_{—}422855 (for polynucleotide and polypeptide, respectively) from Gallus gallus.

According to some embodiments of the invention, the polynucleotide comprises a nucleic acid sequence encoding a polypeptide having at least 80%, at least 81%, 82%, 83%, 84%, 85%, 86%, 87%, 88%, 89%, 90%, 91%, 92%, 93%, 94%, 95%, 96%, 97%, 98%, 99%, e.g., 100% sequence homology or identity to the polypeptide set forth in SEQ ID NO: 27 (GenBank Access No. NP_{—}199588), wherein the polypeptide catalyzes the formation of farnesyl diphosphate (FDP).

According to some embodiments of the invention, the polynucleotide comprises a nucleic acid sequence having at least 80%, at least 81%, 82%, 83%, 84%, 85%, 86%, 87%, 88%, 89%, 90%, 91%, 92%, 93%, 94%, 95%, 96%, 97%, 98%, 99%, e.g., 100% sequence identity to the polynucleotide set forth in SEQ ID NO: 26 (GenBank Accession No. NM_{—}124151), wherein the polynucleotide encodes a polypeptide which catalyzes the formation of farnesyl diphosphate (FDP).

According to some embodiments of the invention, the enzyme in the terpenoid/sterol pathway is expressed in the mitochondria of the yeast cell or is directed to the mitochondria of the yeast cell by a mitochondrial localization signal (as described in further detail above).

According to one embodiment of the invention, the method further comprises exogenously expressing within the yeast cell an enzyme in the terpenoid/sterol pathway, wherein the enzyme is not expressed in, or directed to the mitochondria. Thus, for example, an enzyme in the terpenoid/sterol pathway may further be expressed in a cytosol of a yeast cell.

According to some embodiments of the invention, the method further comprises exogenously expressing within the yeast cell a polynucleotide comprising a nucleic acid sequence encoding a mutated form of yeast 3-hydroxy-3-methylglutaryl-coenzyme A reductase (tHMG) (e.g. EC 1.1.1.34).

Typically, the wild type (normal, non-mutated polypeptide) 3-hydroxy-3-methylglutaryl-co-enzyme-A (HMG-CoA) reductase catalyzes the conversion of HMG-CoA to mevalonate and is one of the early steps in the mevalonic acid (MVA) pathway leading to production of isoprenoids. It is also considered as the rate-limiting enzyme in this pathway in eukaryotic cells. HMGR is an integral membrane protein localized in the endoplasmic reticulum; its N-terminal region consists of a membrane-spanning domain and its catalytically active domain is located in the C-terminal region.

The sequences of the wild type (non-mutated) form of 3-hydroxy-3-methylglutaryl-coenzyme A reductase are known from various organisms including plants (e.g., Artemisia annua), rat, mouse, human, zebrafish, Arabidopsis thaliana, Xenopus laevis, Nasonia vitripennis, Sus scrofa, Andida dubliniensis CD36, Drosophila melanogaster, Macaca mulatta, Salmo solar, Gallus gallus, Bos yaurus, Aedes aegypti, Uncinocarpus reesii 1704, Candida tropicalis MYA-3404, Pediculus humanus corporis, Culex quinquefasciatus, Danio rerio, and more (See via NCBI web site).

For example, the coding sequence of wild type 3-hydroxy-3-methylglutaryl-coenzyme A reductase is provided in GenBank Accession NOs. Q43319 (SEQ ID NO: 21 for polypeptide) and U14625 (SEQ ID NO: 22 for polynucleotide) from Artemisia annua; As well as GenBank Accession NOs. AAB67527 (SEQ ID NO: 23 for polypeptide) and U22382.1 (SEQ ID NO: 24 for polynucleotide).

An N-terminal truncation (e.g., a truncation of amino acids 1-552 of HMG-CoA) removes the membrane-binding region which includes a sterol-sensing domain that is required for feedback regulation and hence forms a soluble deregulated enzyme.

It should be noted that by using the mutated form (hyperactive form) of 3-hydroxy-3-methylglutaryl-coenzyme A reductase the amount of precursors in the MVA pathway (e.g., FDP) increases.

As used herewith the phrase “a mutated form of yeast 3-hydroxy-3-methylglutaryl-coenzyme A reductase (tHMG)” refers to an hyperactive form of 3-hydroxy-3-methylglutaryl-coenzyme A reductase, which comprises the catalytic portion of the enzyme but which is devoid of the domain causing feedback inhibition of the cytosolic mevalonate pathway (MVA) pathway.

Typically, in order to generate the truncated form of HMG-CoA and to prevent feedback inhibition, the membrane spanning domain of the HMG-CoA protein is removed (ca. 500-550 amino acids are removed from the N-terminal portion of the polypeptide), alternatively the sterol-sensing domain contained within this region can be mutated to be non-functional. An exemplary sequence of the N-terminal truncated 3-hydroxy-3-methylglutaryl-coenzyme A reductase is set forth in SEQ ID NOs: 32 and 33 for the polynucleotide and polypeptide sequences, respectively.

According to some embodiments of the invention, the polynucleotide comprises a nucleic acid sequence encoding a polypeptide having at least 80%, at least 81%, 82%, 83%, 84%, 85%, 86%, 87%, 88%, 89%, 90%, 91%, 92%, 93%, 94%, 95%, 96%, 97%, 98%, 99%, e.g., 100% sequence homology or identity to the polypeptide set forth in SEQ ID NO: 33.

According to some embodiments of the invention, the polynucleotide comprises a nucleic acid sequence having at least 80%, at least 81%, 82%, 83%, 84%, 85%, 86%, 87%, 88%, 89%, 90%, 91%, 92%, 93%, 94%, 95%, 96%, 97%, 98%, 99%, e.g., 100% sequence identity to the polynucleotide set forth in SEQ ID NO: 32.

According to one aspect of the present invention, there is provided a method of producing at least one terpene in a yeast cell, the method comprising:

(i) exogenously expressing within the mitochondria of the yeast cell or directing localization thereto a terpene synthase (e.g. an amorphadiene synthase or a valencene synthase);

(ii) exogenously expressing within the mitochondria of the yeast cell or directing localization thereto a farnesyl diphosphate synthase (FDPS); and

(iii) exogenously expressing within the yeast cell a mutated form of yeast 3-hydroxy-3-methylglutaryl-coenzyme A reductase (tHMG), thereby producing the at least one terpene in the yeast cell.

According to some embodiments of the invention, any of the polynucleotides described herein may be comprised in a nucleic acid construct along with a suitable cis acting regulatory element for directing transcription of the nucleic acid sequence in a host cell (e.g., in a yeast cell).

Thus, according to one aspect, there is provided a nucleic acid construct comprising nucleic acid sequence encoding an enzyme selected from the group consisting of a terpene synthase and an enzyme in a terpenoid/sterol pathway which catalyzes formation of a farnesyl diphosphate (FDP), the nucleic acid sequence further comprising at least one cis-acting regulatory element active in a yeast cell for directing expression of the enzyme in the yeast cell and a nucleic acid element for directing expression of the enzyme or localization thereof in the mitochondria of the yeast cell.

According to one embodiment, the nucleic acid construct further comprises exogenously expressing within the yeast cell a polynucleotide comprising a nucleic acid sequence encoding a mutated form of yeast 3-hydroxy-3-methylglutaryl-coenzyme A reductase (tHMG).

The nucleic acid constructs (also referred to herein as an “expression vector”) useful in the methods according to the present invention may be constructed using recombinant DNA technology well known to persons skilled in the art. The gene constructs may be inserted into vectors, which may be commercially available, suitable for transforming into yeast and suitable for expression of the gene of interest in the transformed yeast cells. The nucleic acid constructs of some embodiments of the invention may include additional sequences which render this vector suitable for replication and integration in yeast cells. In addition, a typical cloning vector may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

As used herein, the phrase “cis acting regulatory element” refers to a polynucleotide sequence, preferably a promoter, which binds a trans acting regulator and regulates the transcription of a coding sequence located downstream thereto.

As used herein, the term “promoter” refers to a region of DNA which lies upstream of the transcriptional initiation site of a gene to which RNA polymerase binds to initiate transcription of RNA. The promoter controls where (e.g., which tissue, e.g., which portion of a plant) and/or when (e.g., at which stage or condition in the lifetime of an organism) the gene is expressed.

Any suitable promoter sequence can be used by the nucleic acid construct of the present invention, and exemplary promoters are described hereinunder.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the yeast cells transformed.

According to an embodiment, the promoter in the nucleic acid construct of the present invention is a yeast promoter which serves for directing expression of the exogenous nucleic acid molecule within yeast cells.

As used herein the phrase “yeast promoter” refers to a promoter sequence, including any additional regulatory elements added thereto or contained therein, is at least capable of inducing, conferring, activating or enhancing expression in a yeast cell. Such a promoter can be derived from a yeast cell (e.g. native to the host cell) or may be derived from a plant, bacterial, viral, fungal or animal origin. Such a promoter can be constitutive, i.e., capable of directing high level of gene expression, inducible, i.e., capable of directing gene expression under a stimulus, or chimeric, i.e., formed of portions of at least two different promoters.

Examples of yeast promoters include, without being limited to, cupper inducible promoter CUP1 (P_{CUP1}) as well as promoters for pyruvate decarboxylase (PDC1), phosphoglycerate kinase (PGK), xylose reductase (XR), xylitol dehydrogenase (XDH), L-(+)-lactate-cytochrome c oxidoreductase (CYB2), translation elongation factor-1 (TEF1) and translation elongation factor-2 (TEF2) genes. Additional yeast promoters include the GAP promoter, GAL1 promoter, AOX1 promoter, FLD1 promoter, ADH1 promoter, GAL3 promoter, GAL4 promoter, GAL7 promoter, CTR1 promoter, CTR3 promoter, MET3 promoter and TDH1 promoter.

According to some embodiments of the invention, the nucleic acid construct comprises two or more non identical promoters.

Enhancer elements can be utilized to stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation of the exogenous polynucleotide. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

According to some embodiments of the invention each of the polynucleotides further comprises a terminator sequence for controlling expression of the nucleic acid sequence in the cell (e.g., the yeast cell).

The term “terminator” as used herein refers to an untranslated sequence located downstream (i.e., 3′) to the translation finish codon of a structural gene (generally within about 1 to 1000 bp, more typically 1-500 base pairs and especially 1-100 base pairs) and which controls the end of transcription of the structural gene. Terminator sequences of the present invention may be native to the host cell or exogenous to the cell.

Examples of yeast terminators include, without being limited to, terminators for ADH1, TDH1, pyruvate decarboxylase (PDC1), xylose reductase, (XR), xylitol dehydrogenase (XDH), L-lactate:ferricytochrome c oxidoreductase (CYB2) or iso-2-cytochrome c (CYC) genes [e.g. terminator of CYC1 (T_{CYC1})], or a terminator from the galactose family of genes in yeast, particularly the GAL10 terminator and GAL80 terminator.

It is usually desirable that the vector includes a functional selection marker cassette. When a single deletion construct is used, the marker cassette resides on the vector downstream (i.e., in the 3′ direction) of the 5′ sequence from the target locus and upstream (i.e., in the 5′ direction) of the 3′ sequence from the target locus. Successful transformants will contain the selection marker cassette, which imparts to the successfully transformed cell some characteristic that provides a basis for selection.

A “selection marker gene” is one that encodes a protein needed for the survival and/or growth of the transformed cell in a selective culture medium. Typical selection marker genes encode proteins that (a) confer resistance to antibiotics or other toxins (such genes as, for example, zeocin (Streptoalloteichus hindustanus ble bleomycin resistance gene), G418 (kanamycin-resistance gene of Tn903) or hygromycin (aminoglycoside antibiotic resistance gene from E. coli)), (b) complement auxotrophic deficiencies of the cell (such as, for example, amino acid leucine deficiency (K marxianus LEU2 gene) or uracil deficiency (e.g., K. marxianus or S. cerevisiae URA3 gene)); (c) enable the cell to synthesize critical nutrients not available from simple media, or (d) confer ability for the cell to grow on a particular carbon source (such as a MEL5 gene from S. cerevisiae, which encodes the alpha-galactosidase (melibiase) enzyme and confers the ability to grow on melibiose as the sole carbon source). Preferred selection markers include the zeocin resistance gene, G418 resistance gene, a MEL5 gene and a hygromycin resistance gene.

Examples for yeast expression vectors which may be used in accordance with the present teachings include, but are not limited to, pJ901, pJ902, pJ911, pJ912, pJ1201, pJ1204, pJ1205, pJ1207, pJ1211, pJ1214, pJ1215, pJ1217, pJ1221, pJ1224, pJ1225, pJ1227, pJ1231, pJ1234, pJ1235, pJ1237 which are available from DNA2.0.

Additional yeast expression vectors include TOPO-TA Cloning Vectors, pAO, pDEST, pFLD, pGAP, pPIC, pPink, pTEF, pYES, which are available from Life Technologies.

According to an embodiment, the expression vector comprises the plasmids pRS415, pRS316 and pMY6L for targeted expression in a yeast cell.

In yeast, a number of vectors containing constitutive or inducible promoters can be used, as disclosed in U.S. Pat. Application No: 5,932,447. Alternatively, vectors can be used which promote integration of foreign DNA sequences into the yeast chromosome.

In general, a vector is prepared that contains one or more genes to be inserted and associated promoter and terminator sequences. The vector may contain restriction sites of various types for linearization or fragmentation. Vectors may further contain a backbone portion (such as for propagation in E. coli) many of which are conveniently obtained from commercially available yeast or bacterial vectors.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of the exogenous polynucleotide since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Other than containing the necessary elements for the transcription and translation of the inserted coding sequence, the expression construct of some embodiments of the invention can also include sequences engineered to enhance stability, production, purification, yield or toxicity of the expressed peptide. For example, the expression of a fusion protein or a cleavable fusion protein comprising the protein encoded by the exogenous polynucleotide of some embodiments of the invention (the “exogenous polypeptide” hereinafter) and a heterologous protein can be engineered. Such a fusion protein can be designed so that the fusion protein can be readily isolated by affinity chromatography; e.g., by immobilization on a column specific for the heterologous protein. Where a cleavage site is engineered between the exogenous polypeptide and the heterologous protein, the exogenous polypeptide can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

As mentioned above, the polynucleotide of the present invention may be linked to a nucleic acid element for localization of the enzyme into the mitochondria or for directing expression of the enzyme in the mitochondria of the yeast cell.

Thus, the nucleic acid sequence encoding the enzyme (e.g. terpene synthase or enzyme in a terpenoid/sterol pathway) may be fused in frame to a nucleic acid sequence encoding a mitochondrial signal peptide (e.g. as set forth in SEQ ID NOs: 38, 40, 42, 44, 46, 48, 50 and 52) to thereby direct localization of the enzyme into the mitochondria of the yeast cell.

In addition to the above, the polynucleotide of the present invention can also be introduced into a mitochondria genome thereby enabling mitochondrial expression.

Any method known to one of skill in the art may be used for directing expression of the enzyme (e.g. terpene synthase or enzyme in a terpenoid/sterol pathway) in mitochondria of the yeast cell. Thus, for example, the nucleic acid sequence may be introduced into the yeast cell under the control of promoters operative in mitochondria.

According to one embodiment, expression in the mitochondria is effected by employing a mitochondrion promoter such as mitochondrion specific promoters and/or transcription regulation elements. Examples include, but are not limited to, the ATP6 promoter from tobacco or Arabidopsis mitochondria, the ATP9 promoter from Arabidopsis or tobacco mitochondria or the mitochondrion specific promoter may have a polycistronic “operon” assigned to it, such as the Orf125-NAD3-RSP12 region from tobacco [as described in Sugiyama et al., Mol Gen Genomics (2005) 272: 603-615] or the NAD3-RPS12-Orf299-orf156 region from wheat mitochondria [as described in detail in Clifton et al., Plant Physiol. 136 (3), 3486-3503 (2004)]. Furthermore, the basic yeast mitochondrial promoter consensus sequence: 5′-ATATAAGTA(+1)-3′ may be used alone or in combination with the mitochondrial transcription factor MTF1 [as taught for example in Baoji Xu and David A. Clayton, Nucleic Acids Research (1992) Vol. 20(5) 1053-1059, incorporated herein by reference].

Various methods can be used to introduce the expression vector of some embodiments of the invention into yeast cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

According to one embodiment, the expression vector is introduced into the yeast cell using any transformation or cloning method known to those of skill in the art. Exemplary methods include, but are not limited to, the lithium acetate method, heat shock, spheroplast method, electroporation, biolistic method and glass bead method (all described in detail in Kawai et al. Bioeng Bugs. (2010) 1(6): 395-403, fully incorporated herein by reference). Alternatively, commercial cloning technologies may be used, including but not limited to, Gateway® cloning technology and TOPO® cloning technology both available from Invitrogene.

Thus, for example, the expression vector may be introduced into the yeast cell using the lithium acetate method [as taught by Ito H. et al., Transformation of intact yeast cells treated with alkali cations. J. Bacteriol. (1983) 153:163-168, incorporated herein by reference] as follows: first grow the yeast cells aerobically (e.g. 100 ml of YPD medium at 30° C. with reciprocation). At the mid-log phase, harvest the cells by centrifugation, wash (e.g. once with TE [10 mM Tris-HCl (pH 8.0) and 1.0 mM EDTA] and suspend (e.g. in TE) at a final concentration of about 2×10⁸ cells/ml. Next, to a portion of this cell suspension (e.g. 0.5 ml) add an equal volume of 0.2 M metal ions (LiAc). After 1 h at 30° C. with shaking (e.g. at 140 rpm; stroke, 7.0 cm), incubate 0.1 ml of the cell suspension statically with 15 μl of a plasmid DNA solution (e.g. 670 μg/ml) at 30° C. for 30 min. Next, add an equal volume of 70% PEG 4000 dissolved in water and sterilized at 120° C. for 15 min and mix thoroughly (e.g. on a vortex mixer). After letting the mix stand for 1 h at 30° C., incubate the suspension at 42° C. for 5 min. The cells then need to be cooled immediately to room temperature, washed twice with water, and suspended in 1.0 ml of water. For selecting the yeast transformants, the cell suspension can be directly spread (e.g. 0.1 ml of the cell suspension) on a selective solid medium.

Methods of determining the level in the yeast cell of the RNA transcribed from the exogenous polynucleotide are well known in the art and include, for example, Northern blot analysis, reverse transcription polymerase chain reaction (RT-PCR) analysis (including quantitative, semi-quantitative or real-time RT-PCR) and RNA-in situ hybridization.

Methods of determining the level in the yeast cell of the polypeptide encoded by the exogenous polynucleotide are well known in the art and include, for example, Western blot analysis, activity assay, immunostaining, immunohistochemistry, immunofluoerescence and the like.

According to some embodiments of the invention, the increase in the content of terpene in the plant is compared to the content in native plant grown under the same (e.g., identical) growth conditions.

The terpenes can be analyzed by chromatography, e.g. gas chromatography (GC), mass spectrometry (MS) and/or nuclear magnetic resonance (NMR).

Methods of evaluating an increase in content of terpene are well known in the art and include chromatography, e.g. gas chromatography (GC), mass spectrometry (MS) and/or nuclear magnetic resonance (NMR). Thus, for example, when chromatography-mass spectrometry (GC-MS) analysis is utilized for analysis of terpenoid production, overnight starter culture of yeast (e.g. 5 ml), generated from a stationary culture, may be diluted to an OD₆₀₀ of 0.1 in 10 ml fresh medium supplemented with 100 μM CuSO₄. For in-situ removal of terpenoids a two-phase partitioning batch culture may be employed by adding 10% dodecane as an organic phase. Cultures may then be grown for several days (e.g. 6 days), at which time the organic layer may be sampled for gas chromatography-mass spectrometry (GC-MS) analysis (as described in further detail below).

According to an aspect of some embodiments of the invention, there is provided a yeast cell comprising in a mitochondria thereof an exogenously expressed terpene synthase and/or an exogenously expressed enzyme in a terpenoid/sterol pathway which catalyzes formation of a farnesyl diphosphate (FDP).

According to another embodiment, the yeast cell further comprises an exogenously expressed mutated form of yeast 3-hydroxy-3-methylglutaryl-coenzyme A reductase (tHMG).

According to an aspect of some embodiments of the invention there is provided a method of producing a terpene in a yeast cell, comprising: (a) generating and/or increasing content of the terpene in a yeast cell according to the method of some embodiments of the invention, and (b) isolating the terpene from the yeast cell, thereby producing the terpene.

According to an aspect of some embodiments of the invention there is provided a method of producing a terpene in a yeast cell, comprising: (a) providing a yeast cell according to the method of some embodiments of the invention, and (b) isolating the terpene from the yeast cell, thereby producing the terpene.

According to some embodiments of the invention, the method further comprises providing and/or maintaining conditions suitable for terpene production within the yeast cell, e.g. cultivating the yeast under conditions conducive to the production of the terpene, prior to isolating of the terpene. These conditions are known to the skilled person. Generally, they may be adjusted by selection of an adequate medium, temperature, and pH.

In a further embodiment, the method for producing a terpene comprises the step of isolating the terpene from the medium, from the cells and/or from an organic solvent used for extracting the terpene or in case a two-phase fermentation is performed. The terpene may be isolated by any method used in the art including, but not limited to, chromatography, extraction, in-situ product removal and distillation.

An exemplary method for isolating a terpene comprises purifying same from the cell liquid culture by, for example, in-situ product removal approach (as described in the Examples section which follows). For example, a two-phase partitioning culture may be employed by adding a volume of a biocompatible solvent, e.g. 10%-20% (v/v) n-dodecane, methyl oleate or isopropyl myristate, as the organic phase or a solid adsorbent e.g. Amberlite resin, Diaion HP-20 or activated charcoal.

Once produced and isolated, the purity, content, amount or yield of a terpene can be determined using known methods.

As used herein the term “isolated” with respect to terpene refers to at least partially separated from the cell producing same. In a specific embodiment, isolated refers to free of pathogenic contaminants.

Methods of determining the purity of terpenes are known and in the art, and are also described in the general materials and experimental procedures section of the Examples section which follows.

According to an embodiment, the terpenes are analyzed by chromatography, e.g. gas chromatography (GC), mass spectrometry (MS) and/or nuclear magnetic resonance (NMR).

For example, gas chromatography—mass spectrometry (GC-MS) analysis can be performed using, for example, a Pal autosampler (CTC analytic, Zwingen, switzerland), a TRACE GC 2000 gas chromatograph, and a TRACE DSQ quadrupole mass spectrometer (ThermoFinnigan, Hemel, UK). Gas chromatography may be performed on a 30 m Rtx-5Sil MS column with 0.25 μm film thickness (Restek, Bad Homburg, Germany). The injection temperature is typically set at 250° C., the interface at 280° C., and the ion source adjusted to 200° C. Helium may be used as the carrier gas at a flow rate of 1 ml min⁻¹. The analysis may be performed under the following temperature program: 2 min isothermal heating at 50° C., followed by a 4° C. min⁻¹ oven temperature ramp to 105° C., followed by a 50° C. min⁻¹ oven temperature ramp to 250° C. and a final 5 min heating at 250° C. A scan range of 40 to 450 m/z may be used. Both chromatograms and mass spectra may be evaluated using the XCALIBUR v1.3 program (ThermoFinnigan). Metabolites may be identified by comparing retention time and mass spectra with those of NIST library and to authentic standards when possible (Sigma-Aldrich).

According to some embodiments of the invention, the terpene produced by the method of some embodiments of the invention, from the cell (e.g., from yeast cell) of some embodiments of the invention has a pharmaceutical grade purity of at least about 50%, at least about 55%, at least about 60%, at least about 65%, at least about 70%, at least about 75%, e.g., 80%, 81%, 82%, 83%, 84%, 85%, 86%, 87%, 88%, 89%, 90%, 91%, 92%, 93%, 94%, 95%, 96%, 97%, 98%, 99%, e.g., 100% purity.

According to an aspect of some embodiments of the invention there is provided an isolated terpene produced by the method of some embodiments of the invention.

According to an embodiment of the present invention, there is provided a method of producing a commodity selected from the group consisting of a natural flavor, a food product, a food additive, a fragrance, a cosmetic, a pesticide and a therapeutic agent, comprising producing terpene according to the method of some embodiments of the invention and incorporating the terpene in a process for manufacturing a commodity, thereby producing the commodity.

Thus, the invention can involve the manufacture of a product containing one or more terpenoids, such as one or more terpenoids selected from a hemiterpenoid, a monoterpenoid, a sesquiterpenoid, a diterpenoid, a triterpenoid or a tetraterpenoid.

In some embodiments, the product is a food product, food additive, beverage, chewing gum, candy, or oral care product. In such embodiments, the terpenoid or derivative may be a flavor enhancer or sweetener. For example, the terpenoid or derivative may include one or more of alpha-sinensal; beta-Thuj one; Camphor; Carveol; Carvone; Cineole; Citral; Citronellal; Cubebol; Limonene; Menthol; Menthone; myrcene; Nootkatone; Piperitone; Sabinene hydrate; Steviol; Steviol glycosides; Thymol; Valencene; or a derivative of one or more of such compounds. In other embodiments, the terpenoid or derivative is one or more of alpha, beta and y-humulene; isopinocamphone; (−)-alpha-phellandrene; (+)-1-terpinene-4-ol; (+)-borneol; (+)-verbenone; 1,3,8-menthatriene; 3-carene; 3-Oxo-alpha-Ionone; 4-Oxo-beta-ionone; alpha-sinensal; alpha-terpinolene; alpha-thujene; Ascaridole; Camphene; Carvacrol; Cembrene; E)-4-decenal; Farnesol; Fenchone; gamma-Terpinene; Geraniol; hotrienol; Isoborneol; Limonene; myrcene; nerolidol; ocimene; p-cymene; perillaldehyde; Pulegone; Sabinene; Sabinene hydrate; tagetone; Verbenone; or a derivative of one or more of such compounds.

In some embodiments, the product is a fragrance product, a cosmetic, a cleaning product, or a soap. In such embodiments, the terpenoid or derivative may be a fragrance. For example, the one or more terpenoid or derivative may include one or more of Linalool; alpha-Pinene; Carvone; Citronellal; Citronellol; Citral; Sabinene; Limonene; Verbenone; Geraniol; Cineole; myrcene; Germacrene D; farnesene; Valencene; Nootkatone; patchouli alcohol; Farnesol; beta-Ylangene; .beta.-Santalol; .beta.-Santalene; a-Santalene; .alpha.Santalol; .beta.-vetivone; a-vetivone; khusimol; Sclarene; sclareol; beta-Damascone; beta-Damascenone; or a derivative thereof. In these or other embodiments, the one or more terpenoid or derivative compounds includes one or more of Camphene; Pulegone; Fenchone; Fenchol; Sabinene hydrate; Menthone; Piperitone; Carveol; gamma-Terpinene; beta-Thuj one; dihydro-myrcene; alpha-thujene; alpha-terpineol; ocimene; nerol; nerolidol; E)-4-decenal; 3-carene; (−)-alpha-phellandrene; hotrienol; alpha-terpinolene; (+)-1-terpinene-4-ol; perillaldehyde; verbenone; isopinocamphone; tagetone; trans-myrtanal; alpha-sinensal; 1,3,8-menthatriene; (−)-cis-rose oxide; (+)-borneol; (+)-verbenone; Germacrene A; Germacrene B; Germacrene; Germacrene E; (+)-beta-cadinene; epi-cedrol; alpha, beta and y-humulene; alpha-bisabolene; beta-aryophyllene; Longifolene; alpha-sinensal; alpha-bisabolol; (−).beta.-Copaene; (−)-.alpha.-Copaene; 4(Z),7(Z)-ecadienal; cedrol; cedrene; muuroladiene; isopatchoul-3-ene; isopatchoula-3,5-diene; cedrol; guaiol; (−)-6,9-guaiadiene; bulnesol; guaiol; ledene; ledol; lindestrene; alpha-bergamotene; maaliol; isovalencenol; muurolol T; beta-Ionone; alpha-Ionone; Oxo-Edulan I; Oxo-Edulan II; Theaspirone; Dihydroactinodiolide; 4-Oxoisophorone; Safranal; beta-Cyclocitral; (−)-cis-gamma-irone; (−)-cis-alpha-irone; or a derivative thereof. Such terpenoids and derivatives may be synthesized according to a pathway described above. In some embodiments, the one or more terpenoids include Linalool, which may be synthesized through a pathway comprising one or more of Geranyl pyrophosphate synthase (e.g., AAN01134.1, ACA21458.2), and linalool synthase (e.g., FJ644544, GQ338154, FJ644548).

In some embodiments, the product is a pharmaceutical, and the terpenoid or derivative is an active pharmaceutical ingredient. For example, the terpenoid or derivative may be Artemisinin; Taxol; Taxadiene; levopimaradiene; Gingkolides; Abietadiene; Abietic acid; beta-amyrin; Retinol; or a derivative thereof. In still other embodiments, the terpenoid or derivative is Thymoquinone; Ascaridole; beta-selinene; 5-epi-aristolochene; vetispiradiene; epi-cedrol; alpha, beta and y-humulene; a-cubebene; beta-elemene; Gossypol; Zingiberene; Periplanone B; Capsidiol; Capnellene; illudin; Isocomene; cyperene; Pseudoterosins; Crotophorbolone; Englerin; Psiguadial; Stemodinone; Maritimol; Cyclopamine; Veratramine; Aplyviolene; macfarlandin E; Betulinic acid; Oleanolic acid; Ursoloic acid; Pimaradiene; neo-abietadiene; Squalene; Dolichol; Lupeol; Euphol; Kaurene; Gibberellins; Cassaic acid; Erythroxydiol; Trisporic acid; Podocarpic acid; Retene; Dehydroleucodine; Phorbol; Cafestol; kahweol; Tetrahydrocannabinol; androstenol; or a derivative thereof. Enzymes and encoding genes for synthesizing such terpenoids or derivatives thereof from products of the MEP pathway are known, and some are described above.

In some embodiments, the product is an insecticide, pesticide or pest control agent, and the terpenoid or derivative is an active ingredient. For example, the one or more terpenoid or derivative may include one or more Carvone; Citronellol; Citral; Cineole; Germacrene C; (+)-beta-cadinene; or a derivative thereof. In other embodiments, the one or more terpenoid or derivative is Thymol; Limonene; Geraniol; Isoborneol; beta-Thuj one; myrcene; (+)-verbenone; dimethyl-nonatriene; Germacrene A; Germacrene B; Germacrene D; patchouli alcohol; Guaiazulene; muuroladiene; cedrol; alpha-cadinol; d-occidol; Azadirachtin A; Kaurene; or a derivative thereof. Enzymes and encoding genes for synthesizing such terpenoids or derivatives thereof from products of the MEP pathway are known, and some are described above.

In some embodiments, the product is a cosmetic or personal care product, and the terpenoid or derivative is not a fragrance. For example, one or more terpenoid or derivative is Camphor; Linalool; Carvone; myrcene; farnesene; patchouli alcohol; alpha-bisabolene; alpha-bisabolol; beta-Ylangene; .beta.-Santalol ; .beta.-Santalene; a-Santalene; .alpha.-Santalol; or a derivative thereof. In some embodiments, the terpenoid or derivative is Camphene; Carvacrol; alpha-terpineol; (Z)-beta-ocimene; nerol; (E)-4-decenal; perillaldehyde; (−)-cis-roseoxide; Copaene; 4(Z),7(Z)-decadienal; isopatchoulenone; (−)-6,9-guaiadiene; Retinol; betulin; (−)-cis-gamma-irone; (−)-cis-alpha-irone; Phytoene; Phytofluene; or a derivative thereof. In some embodiments, the one or more terpenoids may include alpha-bisabolene, which may be synthesized through a pathway comprising one or more of farnesyl diphosphate synthase (e.g., AAK63847.1), and bisabolene synthase (HQ343280.1, HQ343279.1).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### GENERAL MATERIALS AND EXPERIMENTAL PROCEDURES

Reagents

Microbial growth medium were purchased from Difco Laboratories (Sparks, Maryland). Molecular biology reagents, enzymes and kits were from Fermentas International (Burlington, Ontario) and Promega (Madison, Wis.). 5-Fluoroorotic acid (5-FOA) was obtained from Zymo Research (Orange, Calif.).

All other chemicals were purchased from Sigma-Aldrich (Rehovot, Israel).

Construction of Yeast Expression Vectors

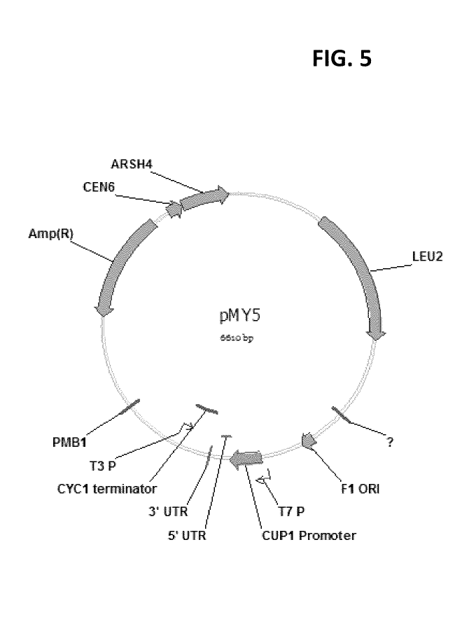

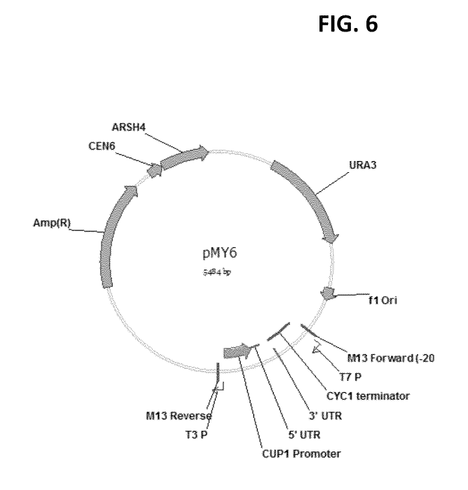

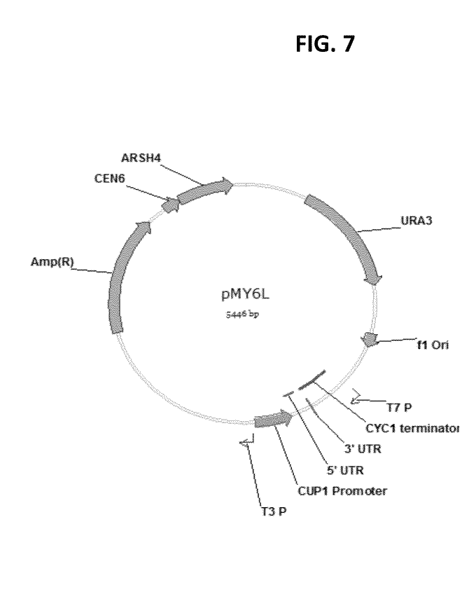

In order to enable controlled expression of several genes in yeast, expression plasmids were constructed. First the promoter and 5′-UTR regions of cupper inducible promoter CUP1 (P_{CUP1}) were amplified by polymerase chain reaction (PCR) from yeast genomic DNA, using primers 1 and 2 (see Table 1, below), and cloned into SacI/NotI digested pRS415 and pRS316 plasmids that carry auxotrophic markers for leucine and uracil [as previously described in Sikorski, R. S. and Hieter, P., Genetics (1989) 122: 19-27]. Next the 3′-UTR and terminator of CYC1 (T_{CYC1}) were PCR amplified, using primers 3 and 4 (see Table 1, below), and ligated into the pRS plasmids carrying P_{CUP1}. The resulting plasmids were termed pMY5 and pMY6 (see FIGS. 5 and 6, respectively), where the number corresponds to the parental pRS plasmids pRS415 and pRS316, respectively. Another plasmid, pMY6L, was built by first deleting P_{CUP1} from pMY6 via digestion with Sad and EcoRI and then introducing back P_{CUP1} into these sites, yielding pMY6L (see FIG. 7) which lacks, as compared to pMY6, NotI, BamHI and SmaI, restriction sites in the polylinker.

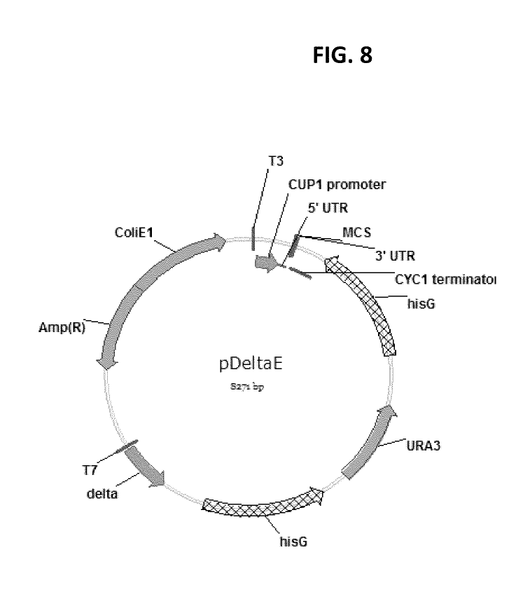

To enable controlled expression of genes following integration into the yeast genome, plasmid pδE was constructed (see FIG. 8). First P_{CUP1} was removed from pMY5, next T_{CYC1} was PCR amplified from pMY5 with primers 5 and 6 (see Table 1, below) that introduced multiple cloning sits. After digestion with NotI and XbaI the two fragments were ligated into SacI/XbaI digested pδ UB plasmid [as previously described in Lee, F. W. F. and Da Silva, N. A., Biotechnol. Prog. (1997) 13: 368-373].

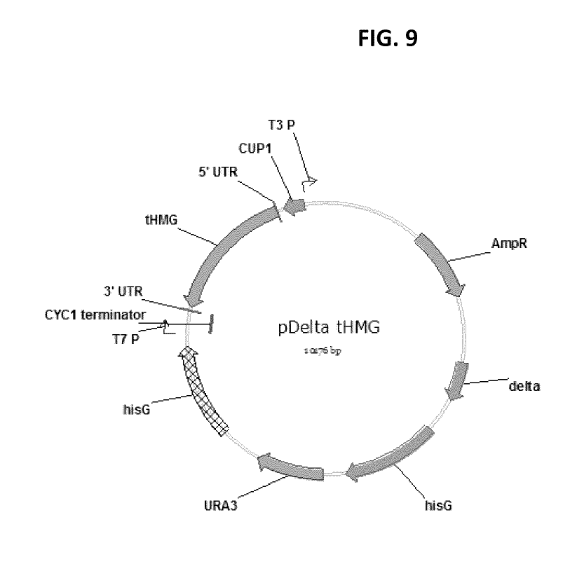

For the generation and expression of a mutated form of yeast hydroxymethylglutaryl CoA reductase (HMG-R) the catalytic domain of HMG1 (tHMG) [GenBank accession Nos: NM_{—}001182434 (SEQ ID NO: 32) for polynucleotide and NP_{—}013636 (SEQ ID NO: 33) for polypeptide] was PCR amplified from yeast genomic DNA using primers 7 and 8 (see Table 1, below) and cloned into EcoRI and XhoI digested pMY6L yielding pMY6L-tHMG. To allow genomic integration of tHMG, plasmid pδ-tHMG was constructed (see FIG. 9). First the multiple cloning sites of plasmid pMY6L were removed by digestion with HindIII and XhoI followed by a T4 DNA polymerase treatment. Self ligation yielded plasmid pMY6L-EcoRI. The tHMG fragment was removed from pMY6L-tHMG by EcoRI and XohI digestion. After T4 DNA polymerase treatment the fragment was cloned into pMY6L-EcoRI digested with EcoRI and blunted with T4 DNA polymerase, yielding pMY6LE-tHMG. The expression cassette including the P_{CUP1}-tHMG-T_{CYC1} was PCR amplified with primer pair 9 and 10 (see Table 1, below) from pMY6LE-tHMG and moved into NotI digested pδ UB.

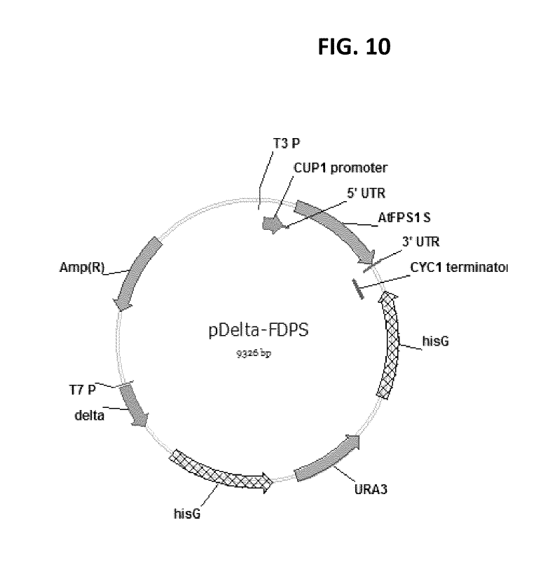

Farnesyl diphosphate synthase (FDPS) was cloned from Arabidopsis thaliana cDNA [GenBank accession Nos: NM_{—}124151 (SEQ ID NO: 26) for polynucleotide and NP_{—}199588 (SEQ ID NO: 27) for polypeptide] using PCR and primer pair 11 and 12 (see Table 1, below), which were designed to target the short cytosolic form (FPS1S) [as was previously described in Cunillera, N. et al., J. Biol. Chem. (1996) 271: 7774-7780; Cunillera, N. et al., J. Biol. Chem. (1997) 272: 15381-15388]. The amplified fragment was subcloned into pGEM-T Easy vector (Promega) (pGEMT-FDPS). For genomic based expression, AtFDPS was removed from pGEMT-FDPS and inserted into EcoRI digested pMY6L-EcoRI, downstream to P_{CUP1}. The expression cassette was removed by cleavage with Sad and KpnI and treatment with T4 DNA polymerase, followed by cloning into XbaI digested and T4 DNA polymerase treated pδ UB integration plasmid, yielding pδ-FDPS (see FIG. 10).

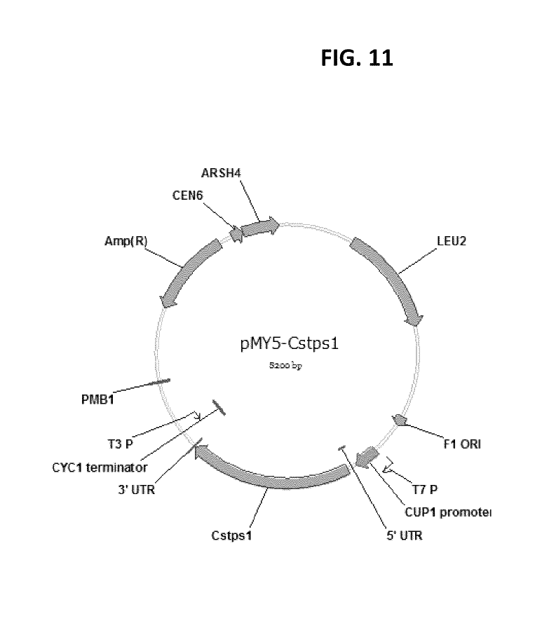

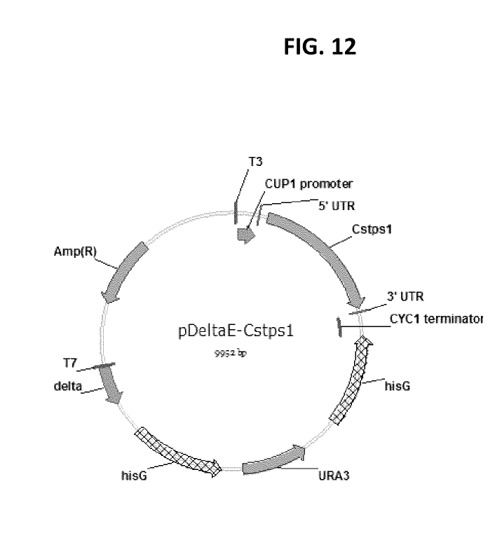

For expression of Citrus sinensis valencene synthase in yeast (Cstps1) the complete coding sequence of Cstps1 [GenBank accession Nos: AF441124 (SEQ ID NO: 28) for polynucleotide and AAQ04608 (SEQ ID NO: 29) for polypeptide] was PCR amplified from pRSETa-Cstps1 [as previously described in Sharon-Asa, L. et al., The Plant Journal (2003) 36: 664-674] with primer pair 13 and 14 (see Table 1, below) and cloned into pMY5 to generate plasmid-based expression cassette or into pδE for genomic expression (FIGS. 11 and 12, respectively).

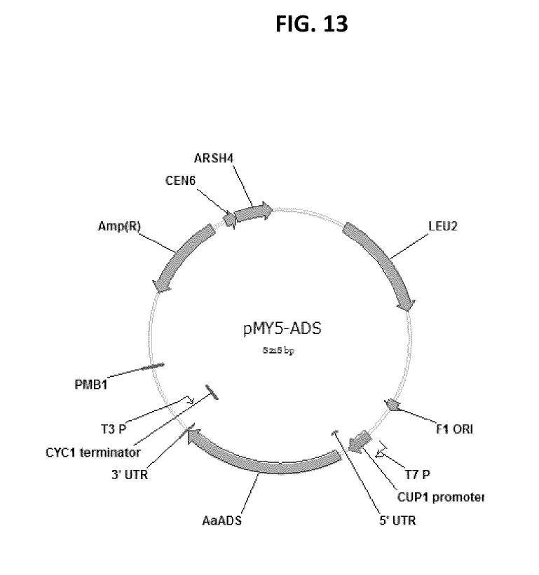

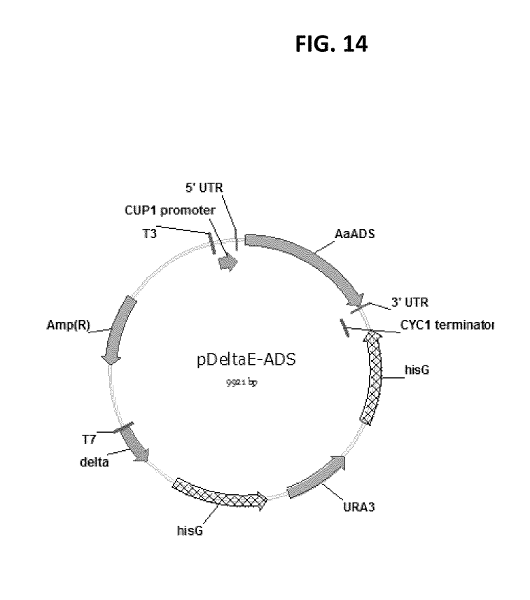

To clone and express in yeast Artemisia annua terpene synthase amorpha-4,11-diene synthase (ADS) total RNA was extracted from A. annua leafs and reverse transcribed to generated the full length ADS cDNA [GenBank accession Nos: ADU25497.1 (SEQ ID NO: 31) for polypeptide and HQ315833.1 (SEQ ID NO: 30) for polynucleotide] using specific primers 15 and 16 (see Table 1, below). After cloning into pGEM-T vector, the coding region was placed into pMY5 episomal vector or into pδE for genomic expression (FIGS. 13 and 14, respectively).

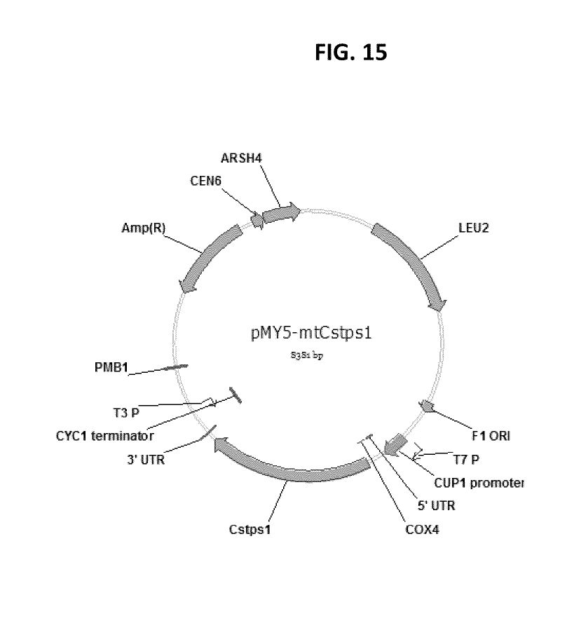

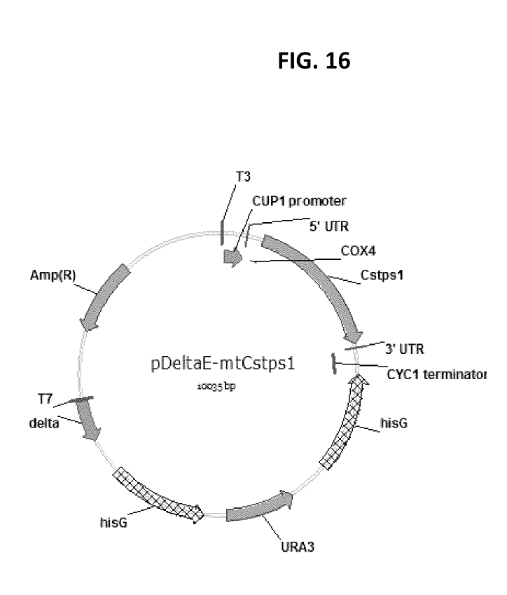

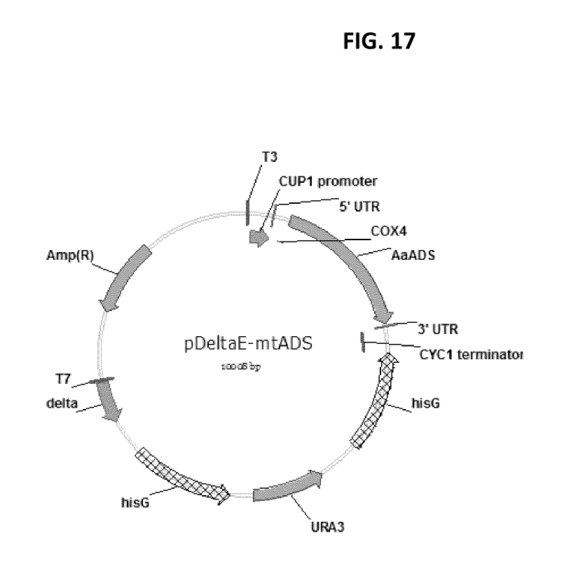

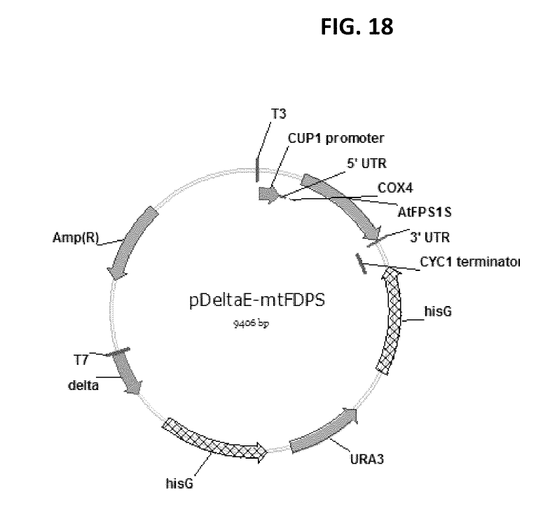

Targeting enzymes of interest (Cstps1, ADS, FDPS) to the yeast mitochondria was achieved using the native yeast mitochondrial signal peptide from COX4 gene (SEQ ID NO: 45) [GenBank Access Nos. NP_{—}011328]. For this, overlap extension PCR was performed using PCR assembly of three oligonucleotides of 60 base pairs each, one of which is complimentary to the 5′ ends of Cstps1 or ADS (see Table 1, below), yielding mtCstp1 and mtADS. To target FDPS, a first PCR was performed on mtADS using primers 17 and 21 (see Table 1, below). The resultant PCR product, primer 12 and pGEMT-FDPS as a template were used to generate mtFDPS fragment in a second PCR. The resulting mitochondrial targeted constructs, mtCstp1, mtADS and mtFDPS were cloned into pMY5 or pδE plasmids, yielding pδE-mtCstp, pδE-mtADS, pδE-mtFDPS (FIGS. 15 to 18, respectively).

TABLE 1
List of primers for construction of yeast
expression vectors

| Primer | |
|---|---|
| number | Sequence (5′ to 3′) * |
| 1 | GCGAGCTCCACCCTTTATTTCAGGCTG |
| | (SEQ ID NO: 1) |
| 2 | ATAGCGGCCGCTTTATGTGATGATTGATTGATTG |
| | (SEQ ID NO: 2) |
| 3 | TAACTCGAGACAGGCCCCTTTTCCTTTG |
| | (SEQ ID NO: 3) |
| 4 | TAGGTACCGCAAATTAAAGCCTTCGAGC |
| | (SEQ ID NO: 4) |
| 5 | ATAGCGGCCGCGTTAACGACGTCGCATGCTGATCAACAGGCCC |
| | CTTTTCCTTTG (SEQ ID NO: 5) |
| 6 | TAATCTAGAGCAAATTAAAGCCTTCGAGC |
| | (SEQ ID NO: 6) |
| 7 | TGAATTCATGGACCAATTGGTGAAAACTGA |
| | (SEQ ID NO: 7) |
| 8 | TACTCGAGTTAGGATTTAATGCAGGTGACG |
| | (SEQ ID NO: 8) |
| 9 | AATGCGGCCGCATGGAGACCGATCTCAAGTC |
| | (SEQ ID NO: 9) |
| 10 | AGCATGCCTACTACTTCTGCCTCTTGTAGATC |
| | (SEQ ID NO: 10) |
| 11 | AAAACAATGTCGTCTGGAGAAACATTTC |
| | (SEQ ID NO: 11) |
| 12 | TCAAAATGGAACGTGGTCTCCTAG |
| | (SEQ ID NO: 12) |
| 13 | ATGGATCCAAAACAATGTCAC TTACAGAAGAA |
| | (SEQ ID NO: 13) |
| 14 | ATCTCGAGTCATATACTCATAGGATAAAC |
| | (SEQ ID NO: 14) |
| 15 | ATGGATCCAAAACAATGCTTTCACTACGTCAATCTATAAGATT |
| | TTTCAAGCCAG (SEQ ID NO: 15) |
| 16 | ATAAGATTTTTCAAGCCAGCCACAAGAACTTTGTGTAGCTCTA |
| | GATATC (SEQ ID NO: 16) |
| 17 | TTGTGTAGCTCTAGATATCTGCTTCAGCAAAAACCCATGTCAC |
| | TTACAGAAGAA (SEQ ID NO: 17) |
| 18 | TTGTGTAGCTCTAGATATCTGCTTCAGCAAAAACCCATGTCGT |
| | CTGGAGAAAC (SEQ ID NO: 18) |
| 19 | AAAGCGGCCGCGTTGATCCA |
| | (SEQ ID NO: 19) |
| 20 | GTTGACTTGAGATCGGTCTCCATGGGTTTTTGCTGAAGCAGAT |
| | ATC (SEQ ID NO: 20) |

Strains, Media and Growth Conditions

All bacterial work was performed with XL1-Blue (Stratagene, La Jolla, Calif.) as a host. Bacteria were grown in Luria-Bertani broth supplemented with 100 mg of ampicillin per ml.

Saccharomyces cerevisiae strains W3031A (MATa, ade2-1, trp1-1, leu2-3,112 h is 3-11,15 ura3-1) and BDXe (developed by the present inventors as a derivative of a commercial strain, generated following screening for uracil auxotrophy by selection on 5-FOA) were used as the parent strains. Yeast were grown in YPD medium (1% yeast extract, 2% peptone, 2% glucose) or synthetic minimal medium (SD; 0.67% yeast nitrogen base, 2% glucose, and auxotrophic amino acids and vitamins as required). Yeasts were transformed by the lithium acetate method; when pδ plasmids were used they were linearized by XhoI digestion prior to transformation. Colonies growing on the relevant drop-out media were verified as harboring the relevant gene by colony PCR [as previously described in Burke, D. et al., Methods in yeast genetics: A Cold Spring Harbor Laboratory course manual. (Cold Spring Harbor Laboratory Press {a}, 2000)]. To allow stacking of genes of interest into the yeast genome, the URA3 selection gene, of an integrated pδ plasmid, was counter selected against by growing cells on medium containing 5-FOA. Retention of the relevant genes following the selection scheme was verified by PCR. Strains used in this work generated following transformation with respective vectors, are listed in Table 2, below.

For analysis of terpenoid production, overnight starter culture of 5 ml, generated from a stationary culture, was diluted to an OD₆₀₀ of 0.1 in 10 ml fresh medium supplemented with 100 μM CuSO₄. For in-situ removal of terpenoids a two-phase partitioning batch culture was employed by adding 10% dodecane as an organic phase. Cultures were grown for 6 days, at which time the organic layer was sampled for gas chromatography-mass spectrometry (GC-MS) analysis. From each transformation event, several colonies were evaluated for farnesol and plant terpenoid productions.

TABLE 2
A description of engineered yeast strains used in the present work

| | | | Plasmid- |
|---|---|---|---|
| | Background | | based |
| Strain | strain | Integration constructs* | constructs |
| M91 | W3031A | | pMY5- |
| | | | Cstps1 |
| M135 | W3031A | δ::P_{CUP1}-tHMG, δ::P_{CUP1}-FDPS | pMY5- |
| | | | Cstps1 |
| M136 | W3031A | | pMY5-ADS |
| M144 | W3031A | δ::P_{CUP1}-tHMG, δ::P_{CUP1}-FDPS, | |
| | | δ::P_{CUP1}-Cstps1 | |
| M201 | W3031A | δ::P_{CUP1}-tHMG, δ::P_{CUP1}-FDPS | pMY5- |
| | | | mtCstps1 |
| M202 | W3031A | δ::P_{CUP1}-tHMG, δ::P_{CUP1}-FDPS, | pMY5- |
| | | δ::P_{CUP1}-Cstps1 | mtCstps1 |
| M208 | W3031A | δ::P_{CUP1}-Cstps1 | |
| M212 | BDXe | δ::P_{CUP1}-Cstps1 | |
| M213 | BDXe | δ::P_{CUP1}-mtADS | |
| M241 | BDXe | δ::P_{CUP1}-mtCstps1 | |
| M242 | BDXe | δ::P_{CUP1}-tHMG, δ::P_{CUP1}-Cstps1 | |
| M243 | BDXe | δ::P_{CUP1}-tHMG, δ::P_{CUP1}- | |
| | | mtCstps1 | |
| M246 | BDXe | δ::P_{CUP1}-tHMG, δ::P_{CUP1}- | |
| | | mtFDPS, δ::P_{CUP1}-mtADS | |
| M263 | BDXe | δ::P_{CUP1}-ADS | |
| M287 | W3031A | δ::P_{CUP1}-tHMG, δ::P_{CUP1}-Cstps1 | |
| M290 | W3031A | δ::P_{CUP1}-FDPS, δ::P_{CUP1}-Cstps1 | |
| M1057 | BDXe | δ::P_{CUP1}-tHMG, δ::P_{CUP1}-FDPS, | |
| | | δ::P_{CUP1}-ADS | |
| M1058 | BDXe | δ::P_{CUP1}-tHMG, δ::P_{CUP1}-FDPS, | |
| | | δ::P_{CUP1}-mtADS | |
| M1059 | BDXe | δ::P_{CUP1}-tHMG, δ::P_{CUP1}- | |
| | | mtFDPS, δ::P_{CUP1}-ADS | |

*δ:: denotes integration into a δ element insertion site using a pδ UB or pδE vector

Gas Chromatography—Mass Spectrometry (GC-MS) Analysis

From each sample 1 μl of dodecane was analyzed by GC-MS. The system was composed of a Pal autosampler (CTC analytic, Zwingen, switzerland), a TRACE GC 2000 gas chromatograph, and a TRACE DSQ quadrupole mass spectrometer (ThermoFinnigan, Hemel, UK). Gas chromatography was performed on a 30 m Rtx-5Sil MS column with 0.25 μm film thickness (Restek, Bad Homburg, Germany). The injection temperature was set at 250° C., the interface at 280° C., and the ion source adjusted to 200° C. Helium was used as the carrier gas at a flow rate of 1 ml min⁻¹. The analysis was performed under the following temperature program: 2 min isothermal heating at 50° C., followed by a 4° C. min-1 oven temperature ramp to 105° C., followed by a 50° C. min-1 oven temperature ramp to 250° C. and a final 5 min heating at 250° C. A scan range of 40 to 450 m/z was used. Both chromatograms and mass spectra were evaluated using the XCALIBUR v1.3 program (ThermoFinnigan). Metabolites were identified by comparing retention time and mass spectra with those of NIST library and to authentic standards when possible (Sigma-Aldrich).

### Example 1

### Production of Valencene and Amorpha-4,11-Diene in Yeast

Valencene synthase (Cstps1) and amorpha-4,11-diene synthase (ADS) were cloned into pMY5 episomal vector or pδE vector for genomic expression downstream to the copper inducible CUP1 promoter. The vectors were transformed into the W3031A yeast strain and sesquiterpenes' productions were evaluated by GC-MS analysis. Upon induction of TPSs expression, from either vector, valencene and amorphadiene were readily identified in the dodecane extracts of yeast cultures expressing Cstps1 (FIG. 1A) or ADS (FIG. 1B), respectively, and not in a control lines (FIGS. 1A-B). The identity of the terpenoids was verified by comparisons of retention time (RT) and MS to those of NIST library and to authentic standard (for valencene) or to amorpha-4,11-diene from a hexanolic extract of A. annua leaf tissues. Similar results were obtained when BDXe strain was used (FIGS. 3B and 2B), albeit higher titers of the terpenoids were obtained (compare M208 in FIG. 2A with M212 in FIG. 3B).

### Example 2

### Metabolic Engineering the Mevalonic Acid Pathway Enhanced Plant Valencene and Amorpha-4,11-Diene Production in Yeast

To facilitate high production levels of plant terpenoids, produced by yeast expressing ADS or Cstps1, the flux in the native yeast MVA pathway was elevated. HMG-R is the main rate-limiting step in this pathway and its activity is regulated by feedback inhibition [Gardner R. G. and Hampton R. Y., J. Biol. Chem. (1999) 274: 31671-31678]. Therefore, a mutated HMG-R enzyme was generated to overcome the negative regulation [Donald K. A. et al., Appl. Environ. Microbiol. (1997) 63: 3341-3344], and expressed it, using the integration plasmid pδ-tHMG, in yeast under the control of a strong promoter. Upon co-expression of tHMG and Cstps1 in the same W3031A yeast background, as compared to Cstps1 alone, up to 1.5-fold higher levels of valencene were produced as determined by GC-MS analysis (FIG. 2A). An even stronger effect on valencene production was observed when another suggested rate-limiting step in the pathway, was overcome by expressing FDPS cloned from A. thaliana plants: about 3-fold increase in production of valencene was measured in the yeast cells with FDPS and Cstps1 as compared to yeast with Cstps1 only (FIG. 2A). Moreover the effect of tHMG and FDPS was additive and combination of both genes with Cstps1 led to additional increase in valencene levels as compared to Cstps1 with either tHMG or FDPS (FIG. 2A). To verify that MVA pathway engineering enhances production of plant terpenoids other than valencene and is not exclusive to W3031A strain, BDXe yeast strain was engineered, already transformed with pδE-ADS, with pδ-tHMG and pδ-FDPS. Similarly to valencene in W3031A, production levels of amorphadiene were increased by 1.5-fold by addition of the tHMG and the FDPS, as indicated by GC-MS analysis (FIG. 2B).

### Example 3

### Targeting Plant Terpene Synthases to the Yeast Mitochondria Highly Elevated Terpenoids Production

The present inventors speculated that a viable farnesyl diphosphate (FDP) pool is present in the yeast mitochondria, as at least three enzymes that utilize FDP are present in this organelle, namely COX10, COQ1 and BTS1 responsible for the synthesis of the isoprenoid chain of Heme A, ubiquinone and GGDP, respectively (FIG. 19). To test whether this pool can be harnessed for the synthesis of heterologous terpenoids by TPSs, the bona fide mitochondrial targeting signal peptide from the yeast COX4 gene (SEQ ID NO: 45) was fused to Cstps1 and to ADS (generating mtCstps1 and mtADS). The new constructs were inserted into pMY5 or pδE yeast expression vectors and transformed into yeast. GC-MS analysis of terpenoids produced by strain M201, carrying pMY5-mtCstps1 in the W3031A background engineered with tHMG and FDPS, revealed a 5-fold increase in valencene levels as compared to strain M135 expressing the native form of Cstps1 from pMY5 and in the same genetic background (FIG. 3A). Co-expression of both a genome integrated copy (from pδE) of the valencene synthase and an episomal copy (from pMY5) of mitochondrial targeted Cstps1 led to an 1.5-fold increase in valencene production levels, compared to integrated copy of Cstps1 only (FIG. 3A, M202 verses M144). These results were validated in the BDXe yeast strain background: pδE-mtCstps1 vector was used to generate cells expressing the mitochondrial targeted Cstps1 in both the WT and tHMG expressing BDXe backgrounds. Similarly to W3031A, targeting of valencene synthase to BDXe mitochondria, as compared to cytosol, elevated valencene production levels by ca. 3-fold (M242 vs M212 in FIG. 3B), furthermore production of valencene could be further boosted by introducing also tHMG (M241, FIG. 3B), as can be seen from quantitative GC-MS analysis (FIG. 3B).

To evaluate the effect of targeting ADS to the mitochondria in BDXe yeast background, pδE-mtADS vector was transformed into WT BDXe or BDXe strains containing tHMG and FDPS, all under the control of P_{CUP1}. GC-MS analysis of the sesquiterpenes accumulated in these strains revealed that, as with Cstps1, targeting of ADS terpene synthase to the yeast mitochondria, as compared to cytosol, elevated amorphadiene biosynthesis (FIG. 3C). The effect of mtADS compared to ADS was more pronounced then the effect of targeting Cstps1 to the mitochondria: a ca. 8-fold increase in amorphadiene level was observed in mtADS versus ADS strains (M213 vs M263, FIG. 3C). Enhancement of the metabolic flux in the yeast MVA pathway via expression of tHMG and FDPS together with mtADS further elevated the production levels of amorphadiene yielding ca. 2.6 mg/l medium.

### Example 4

### Targeting Farnesyl Diphosphate Synthase Together with TPSs to the Yeast Mitochondria Enhances Production of Terpenoids

Yeast Erg20p, the native yeast FDPS, does not seem to be targeted to the mitochondria, unlike one of the isoforms of plant FDPS. To test if the mitochondrial targeting of FDPS in yeast can elevate production levels of terpenes of interest, driven by mtTPSs, a mitochondria-targeted FDPS was generated via fusion to COX4 targeting signal. The resultant mtFDPS was transformed into BDXe strains already engineered with tHMG and mtADS. For comparison, mtFDPS was also introduced into BDXe strain containing tHMG and ADS. GC-MS analysis and monitoring of terpenoids' accumulation in cultures of these strains reveled that mtFDPS enhanced amorphadiene levels driven by mtADS, as compared to ADS, by ca. 9 fold (M246 vs M1059, FIG. 4). Replacing FDPS with mtFDPS in BDXe containing tHMG and mtADS increased amorphadiene levels by ca. 1.5-fold (M246 vs M1058). Overall, as compared to BDXe producing amorphadiene driven by ADS, ca. 17 fold increase in the production levels of amorphadiene was achieved by employing mtFDPS, tHMG and mtADS in the BDXe background (M246 vs M263, FIGS. 3A-C and FIG. 4).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of producing at least one terpene in a yeast cell, the method comprising exogenously expressing within the mitochondria of the yeast cell or directing localization thereto a terpene synthase, thereby producing the at least one terpene in the yeast cell.

2. The method of claim 1, wherein said terpene synthase is translationally fused to a mitochondrial localization signal (MLS) peptide.

3. The method of claim 1, further comprising exogenously expressing within the yeast cell an enzyme in a terpenoid/sterol pathway which catalyzes formation of a farnesyl diphosphate (FDP).

4. The method of claim 3, wherein said exogenously expressing within the yeast cell said enzyme in said terpenoid/sterol pathway which catalyzes formation of said farnesyl diphosphate is effected in the mitochondria of the yeast cell or by directing localization of said enzyme to said mitochondria of the yeast cell.

5. The method of claim 4, wherein said enzyme in said terpenoid/sterol pathway is translationally fused to a mitochondrial localization signal (MLS) peptide.

6. The method of claim 1, further comprising exogenously expressing within the yeast cell a mutated form of yeast 3-hydroxy-3-methylglutaryl-coenzyme A reductase (tHMG).

7. The method of claim 1, further comprising exogenously expressing within the yeast cell a terpene synthase, wherein said terpene synthase is not expressed in, or directed to said mitochondria.

8. The method of claim 1, wherein said terpene synthase is selected from the group consisting of a valencene synthase, a linalool synthase, a phytoene synthase, an amorphadiene synthase, a limonene synthase and a taxadiene synthase.

9. The method of claim 3, wherein said enzyme in said terpenoid/sterol pathway is selected from the group consisting of a geranyl diphosphate synthase, a farnesyl diphosphate synthase and a geranylgeranyl diphosphate synthase.

10. The method of claim 1, wherein said at least one terpene is a plant terpene.

11. The method of claim 1, wherein said at least one terpene is a sesquiterpene.

12. The method of claim 1, wherein said at least one terpene is selected from the group consisting of a sesquiterpene, a hemiterpene, a monoterpene, a diterpene, a sesterterpene, a triterpene, a sesquarterpene, a tetraterpene and a polyterpene.

13. The method of claim 1, wherein said at least one terpene is selected from the group consisting of a taxadiene, a linalool, a valencene, a phytoene, an amorpha-4,11-diene, a limonene and a farnesyl diphosphate.

14. A method of producing at least one terpene in a yeast cell, the method comprising:
(i) exogenously expressing within the mitochondria of the yeast cell or directing localization thereto a terpene synthase;
(ii) exogenously expressing within the mitochondria of the yeast cell or directing localization thereto a farnesyl diphosphate synthase; and
(iii) exogenously expressing within the yeast cell a mutated form of yeast 3-hydroxy-3-methylglutaryl-coenzyme A reductase (tHMG),
thereby producing the at least one terpene in the yeast cell.

15. The method of claim 14, wherein said terpene synthase and/or said farnesyl diphosphate synthase is translationally fused to a mitochondrial localization signal (MLS) peptide.

16. The method of claim 14, wherein said terpene synthase comprises an amorphadiene synthase.

17. The method of claim 14, wherein said terpene synthase comprises a valencene synthase.

18. The method of claim 14, further comprising exogenously expressing within the yeast cell a terpene synthase, wherein said terpene synthase is not expressed in or directed to said mitochondria.

19. A nucleic acid construct, comprising a nucleic acid sequence encoding an enzyme selected from the group consisting of a terpene synthase and an enzyme in a terpenoid/sterol pathway which catalyzes formation of a farnesyl diphosphate (FDP), said nucleic acid sequence further comprising at least one cis-acting regulatory element active in a yeast cell for directing expression of said enzyme in the yeast cell and a nucleic acid element for directing expression of said enzyme or localization thereof in the mitochondria of the yeast cell.

20. The nucleic acid construct of claim 19, wherein said nucleic acid element encodes a mitochondrial signal peptide fused in frame to said enzyme.

21. The nucleic acid construct of claim 19, further comprising a nucleic acid sequence encoding a mutated form of yeast 3-hydroxy-3-methylglutaryl-coenzyme A reductase (tHMG).

22. A yeast cell comprising in a mitochondria thereof an exogenously expressed terpene synthase and/or an exogenously expressed enzyme in a terpenoid/sterol pathway which catalyzes formation of a farnesyl diphosphate (FDP).

23. The yeast cell of claim 22, wherein said yeast cell further comprises an exogenously expressed mutated form of yeast 3-hydroxy-3-methylglutaryl-coenzyme A reductase (tHMG).

24. A method of producing terpene in a yeast cell, comprising:
(a) generating and/or increasing content of at least one terpene in said yeast cell according to the method of claim 1; and
(b) isolating the terpene from said yeast cell,
thereby producing the terpene.

25. A method of producing terpene in a yeast cell, comprising:
(a) generating and/or increasing content of at least one terpene in said yeast cell according to the method of claim 14; and
(b) isolating the terpene from said yeast cell,
thereby producing the terpene.

26. A method of producing terpene in a yeast cell, comprising:
(a) providing said yeast cell of claim 22, and
(b) isolating the terpene from said yeast cell,
thereby producing the terpene.

27. An isolated terpene produced by the method of claim 24.

28. A method of producing a commodity selected from the group consisting of a natural flavor, a food product, a food additive, a fragrance, a cosmetic, a later/rubber, a fuel, a pesticide and a therapeutic agent, comprising producing terpene according to claim 24 and incorporating said terpene in a process for manufacturing a commodity, thereby producing said commodity.


SEQUENCE LISTING (52 sequences)

SEQ ID NO: 1 - DNA, 27 bp, Artificial sequence
  Single strand DNA oligonucleotide
gcgagctccaccctttatttcaggctg

SEQ ID NO: 2 - DNA, 34 bp, Artificial sequence
  Single strand DNA oligonucleotide
atagcggccgctttatgtgatgattgattgattg

SEQ ID NO: 3 - DNA, 28 bp, Artificial sequence
  Single strand DNA oligonucleotide
taactcgagacaggccccttttcctttg

SEQ ID NO: 4 - DNA, 28 bp, Artificial sequence
  Single strand DNA oligonucleotide
taggtaccgcaaattaaagccttcgagc

SEQ ID NO: 5 - DNA, 54 bp, Artificial sequence
  Single strand DNA oligonucleotide
atagcggccgcgttaacgacgtcgcatgctgatcaacaggccccttttcctttg

SEQ ID NO: 6 - DNA, 29 bp, Artificial sequence
  Single strand DNA oligonucleotide
taatctagagcaaattaaagccttcgagc

SEQ ID NO: 7 - DNA, 30 bp, Artificial sequence
  Single strand DNA oligonucleotide
tgaattcatggaccaattggtgaaaactga

SEQ ID NO: 8 - DNA, 30 bp, Artificial sequence
  Single strand DNA oligonucleotide
tactcgagttaggatttaatgcaggtgacg

SEQ ID NO: 9 - DNA, 31 bp, Artificial sequence
  Single strand DNA oligonucleotide
aatgcggccgcatggagaccgatctcaagtc

SEQ ID NO: 10 - DNA, 32 bp, Artificial sequence
  Single strand DNA oligonucleotide
agcatgcctactacttctgcctcttgtagatc

SEQ ID NO: 11 - DNA, 28 bp, Artificial sequence
  Single strand DNA oligonucleotide
aaaacaatgtcgtctggagaaacatttc

SEQ ID NO: 12 - DNA, 24 bp, Artificial sequence
  Single strand DNA oligonucleotide
tcaaaatggaacgtggtctcctag

SEQ ID NO: 13 - DNA, 32 bp, Artificial sequence
  Single strand DNA oligonucleotide
atggatccaaaacaatgtcacttacagaagaa

SEQ ID NO: 14 - DNA, 29 bp, Artificial sequence
  Single strand DNA oligonucleotide
atctcgagtcatatactcataggataaac

SEQ ID NO: 15 - DNA, 54 bp, Artificial sequence
  Single strand DNA oligonucleotide
atggatccaaaacaatgctttcactacgtcaatctataagatttttcaagccag

SEQ ID NO: 16 - DNA, 49 bp, Artificial sequence
  Single strand DNA oligonucleotide
ataagatttttcaagccagccacaagaactttgtgtagctctagatatc

SEQ ID NO: 17 - DNA, 54 bp, Artificial sequence
  Single strand DNA oligonucleotide
ttgtgtagctctagatatctgcttcagcaaaaacccatgtcacttacagaagaa

SEQ ID NO: 18 - DNA, 53 bp, Artificial sequence
  Single strand DNA oligonucleotide
ttgtgtagctctagatatctgcttcagcaaaaacccatgtcgtctggagaaac

SEQ ID NO: 19 - DNA, 20 bp, Artificial sequence
  Single strand DNA oligonucleotide
aaagcggccgcgttgatcca

SEQ ID NO: 20 - DNA, 46 bp, Artificial sequence
  Single strand DNA oligonucleotide
gttgacttgagatcggtctccatgggtttttgctgaagcagatatc

SEQ ID NO: 21 - protein, 560 aa, Artemisia annua
MDLRRKLPPKPPSSTTTKQPSHRSHSPTPIPKASDALPLPLYLTNTFFFTLFFSVAYYLLHRWRDKIRSGTPLHVVTLTELSAIVLLIASFIYLLGFFGIDFVQSFISRENEQLNNDDHNVISTNNVLSDRRLVYDYDGFDNDDDVIVKSVVSGEVNSYSLEASLGDCYRAAKIRRRAVERIVGREVLGLGFEGFDYESILGQCCEMPIGYVQVPVGVAGPLLLNGGEFMVPMATTEGCLVASTNRGCKAICLSGGATAILLKDGMTRAPVVRFATAERASQLKFYLEDGVNFDTLSVVFNKSSRFARLQNIQCSIAGKNLYIRFTCSTGDAMGMNMVSKGVQNVLDFLQNDFPDMDVIGISGNFCSDKKPAAVNWIEGRGKSVVCEAVITEEVVRKVLKTTVPALVELNMLKNLTGSAIAGSLGGFNAHAANIVSAVFIATGQDPAQNIESSHCITMMEAVNNGKDLHVSVTMPSIEVGTVGGGTQLASQSACLNLLGVKGACIESPGSNAQLLARIVAGSVLAGELSLMSAISAGQLVKSHMKYNRSSRDMSAIASKV

SEQ ID NO: 22 - DNA, 1683 bp, Artemisia annua
atggatctccgtcgtaaactaccacccaaaccaccatcatcaacaaccaccaaacaaccgtcacaccgctcacattcaccaacaccaataccaaaagcatcagacgcattaccattaccattatacctaaccaacaccttcttcttcaccttattcttctcagtcgcttactatctccttcacagatggcgcgacaagatccgttccggcacgccgttgcacgtcgtcacgttaactgaattatctgctattgttttactcattgcttcctttatttatttgttaggtttctttggtattgattttgtccagtcgtttatttcgcgcgaaaacgaacaattgaataatgatgatcataatgttattagtactaataatgtgttgtctgatagaaggcttgtttatgattatgatggatttgataatgatgatgatgtgattgtgaagagtgttgttagtggtgaggtgaattcgtattcgttagaggcgagtttaggtgattgttatagagcggctaagatacgtagacgtgcggttgagaggattgtagggagggaggttttagggttagggtttgaggggtttgattacgagagtattttagggcagtgttgtgagatgcctataggttatgttcaggtgccggtgggggtagcggggcctttgttgttgaatggcggggagtttatggtgcctatggctactacggaagggtgtttggttgctagtacgaatagagggtgtaaggcgatatgtttgtccggtggggcgactgcgattttgttgaaagatgggatgactagagcgcctgttgttaggtttgccactgcggagagggcttcacagttgaagttttatttggaagatggggtgaattttgacacgttgagtgtcgttttcaataaatcaagcagatttgctaggctccaaaatattcaatgctcaattgccggaaagaatctatatatcagatttacttgcagcacgggtgatgcaatgggaatgaacatggtgtcaaagggtgtccaaaatgtgttggattttcttcaaaatgatttcccagacatggatgtgattggtatatctggaaatttctgttcggataaaaaacccgctgcagttaattggattgaggggcgtggaaaatctgttgtgtgcgaggcagtaatcactgaagaggttgtgagaaaagtgcttaaaaccacagtacctgcacttgtagaacttaacatgcttaagaaccttactggttccgctattgctggttctcttggtggatttaatgcacatgctgcaaatatcgtatctgcagtctttatagccactggtcaggatccggcccaaaacattgagagctctcactgcataactatgatggaagctgtcaataatggaaaagatctgcacgtatctgttaccatgccttcaatagaggttggcacagttggaggagggacacaattagcatcacaatcagcatgcttgaacctacttggagtcaagggtgcgtgcatagaatcaccaggctcaaacgctcaattgctagcaaggatagttgctggttcggtgttggctggtgaattgtcgttgatgtctgccatatcagctgggcagttggttaaaagccatatgaaatacaacagatcaagcagagacatgtcagcaattgcgtcaaaggtgtga

SEQ ID NO: 23 - protein, 1045 aa, Saccharomyces cerevisiae
MSLPLKTIVHLVKPFACTARFSARYPIHVIVVAVLLSAAAYLSVTQSYLNEWKLDSNQYSTYLSIKPDELFEKCTHYYRSPVSDTWKLLSSKEAADIYTPFHYYLSTISFQSKDNSTTLPSLDDVIYSVDHTRYLLSEEPKIPTELVSENGTKWRLRNNSNFILDLHNIYRNMVKQFSNKTSEFDQFDLFIILAAYLTLFYTLCCLFNDMRKIGSKFWLSFSALSNSACALYLSLYTTHSLLKKPASLLSLVIGLPFIVVIIGFKHKVRLAAFSLQKFHRISIDKKITVSNIIYEAMFQEGAYLIRDYLFYISSFIGCAIYARHLPGLVNFCILSTFMLVFDLLLSATFYSAILSMKLEINIIHRSTVIRQTLEEDGVVPTTADIIYKDETASEPHFLRSNVAIILGKASVIGLLLLINLYVFTDKLNATILNTVYFDSTIYSLPNFINYKDIGNLSNQVIISVLPKQYYTPLKKYHQIEDSVLLIIDSVSNAIRDQFISKLLFFAFAVSISINVYLLNAAKIHTGYMNFQPQSNKIDDLVVQQKSATIEFSETRSMPASSGLETPVTAKDIIISEEIQNNECVYALSSQDEPIRPLSNLVELMEKEQLKNMNNTEVSNLVVNGKLPLYSLEKKLEDTTRAVLVRRKALSTLAESPILVSEKLPFRNYDYDRVFGACCENVIGYMPIPVGVIGPLIIDGTSYHIPMATTEGCLVASAMRGCKAINAGGGATTVLTKDGMTRGPVVRFPTLIRSGACKIWLDSEEGQNSIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVKSVLKSDVSALVELNISKNLVGSAMAGSVGGFNAHAANLVTALFLALGQDPAQNVESSNCITLMKEVDGDLRISVSMPSIEVGTIGGGTVLEPQGAMLDLLGVRGPHPTEPGANARQLARIIACAVLAGELSLCSALAAGHLVQSHMTHNRKTNKANELPQPSNKGPPCKTSALL

SEQ ID NO: 24 - DNA, 3138 bp, Saccharomyces cerevisiae
atgtcacttcccttaaaaacgatagtacatttggtaaagccctttgcttgcactgctaggtttagtgcgagatacccaatccacgtcattgttgttgctgttttattgagtgccgctgcttatctatccgtgacacaatcttaccttaacgaatggaagctggactctaatcagtattctacatacttaagcataaagccggatgagttgtttgaaaaatgcacacactactataggtctcctgtgtctgatacatggaagttactcagctctaaagaagccgccgatatttataccccttttcattattatttgtctaccataagttttcaaagtaaggacaattcaacgactttgccttcccttgatgacgttatttacagtgttgaccataccaggtacttattaagtgaagagccaaagataccaactgaactagtgtctgaaaacggaacgaaatggagattgagaaacaacagcaattttattttggacctgcataatatttaccgaaatatggtgaagcaattttctaacaaaacgagcgaatttgatcagttcgatttgtttatcatcctagctgcttaccttactcttttttatactctctgttgcctgtttaatgacatgaggaaaatcggatcaaagttttggttaagcttttctgctctttcaaactctgcatgcgcattatatttatcgctgtacacaactcacagtttattgaagaaaccggcttccttattaagtttggtcattggactaccatttatcgtagtaattattggctttaagcataaagttcgacttgcggcattctcgctacaaaaattccacagaattagtattgacaagaaaataacggtaagcaacattatttatgaggctatgtttcaagaaggtgcctacttaatccgcgactacttattttatattagctccttcattggatgtgctatttatgctagacatcttcccggattggtcaatttctgtattttgtctacatttatgctagttttcgacttgcttttgtctgctactttttattctgccattttatcaatgaagctggaaattaacatcattcacagatcaaccgtcatcagacagactttggaagaggacggagttgtcccaactacagcagatattatatataaggatgaaactgcctcagaaccacattttttgagatctaacgtggctatcattctgggaaaagcatcagttattggtcttttgcttctgatcaacctttatgttttcacagataagttaaatgctacaatactaaacacggtatattttgactctacaatttactcgttaccaaattttatcaattataaagatattggcaatctcagcaatcaagtgatcatttccgtgttgccaaagcaatattatactccgctgaaaaaataccatcagatcgaagattctgttctacttatcattgattccgttagcaatgctattcgggaccaatttatcagcaagttacttttttttgcatttgcagttagtatttccatcaatgtctacttactgaatgctgcaaaaattcacacaggatacatgaacttccaaccacaatcaaataagatcgatgatcttgttgttcagcaaaaatcggcaacgattgagttttcagaaactcgaagtatgcctgcttcttctggcctagaaactccagtgaccgcgaaagatataattatctctgaagaaatccagaataacgaatgcgtctatgctttgagttcccaggacgagcctatccgtcctttatcgaatttagtggaacttatggagaaagaacaattaaagaacatgaataatactgaggtttcgaatcttgtcgtcaacggtaaactgccattatattccttagagaaaaaattagaggacacaactcgtgcggttttagttaggagaaaggcactttcaactttggctgaatcgccaattttagtttccgaaaaattgcccttcagaaattatgattatgatcgcgtttttggagcttgctgtgaaaatgtcatcggctatatgccaataccagttggtgtaattggtccattaattattgatggaacatcttatcacataccaatggcaaccacggaaggttgtttagtggcttcagctatgcgtggttgcaaagccatcaatgctggtggtggtgcaacaactgttttaaccaaagatggtatgactagaggcccagtcgttcgtttccctactttaataagatctggtgcctgcaagatatggttagactcggaagagggacaaaattcaattaaaaaagcttttaattctacatcaaggtttgcacgtttgcaacatattcaaacctgtctagcaggcgatttgctttttatgagatttcggacaactaccggtgacgcaatgggtatgaacatgatatcgaaaggtgtcgaatactctttgaaacaaatggtagaagaatatggttgggaagatatggaagttgtctccgtatctggtaactattgtactgataagaaacctgccgcaatcaattggattgaaggtcgtggtaaaagtgtcgtagctgaagctactattcctggtgatgtcgtaaaaagtgttttaaagagcgatgtttccgctttagttgaattaaatatatccaagaacttggttggatccgcaatggctggatctgttggtggtttcaacgcgcacgcagctaatttggtcactgcacttttcttggcattaggccaagatcctgcgcagaacgtcgaaagttccaactgtataactttgatgaaggaagttgatggtgatttaaggatctctgtttccatgccatctattgaagttggtacgattggcgggggtactgttctggagcctcagggcgccatgcttgatcttctcggcgttcgtggtcctcaccccactgaacctggagcaaatgctaggcaattagctagaataatcgcgtgtgctgtcttggctggtgaactgtctctgtgctccgcacttgctgccggtcacctggtacaaagccatatgactcacaaccgtaaaacaaacaaagccaatgaactgccacaaccaagtaacaaagggcccccctgtaaaacctcagcattattataa

SEQ ID NO: 25 - protein, 74 aa, Artificial sequence
  Amino acid sequence of the  Presequence of the subunit 9 of the F0 ATPase of Neurospora crassa
MIQVAKIIGTGLATTGLIGAGIGIGVVFGSLIIGVSRNPSLKSQLFAYAILGFAFSEATGLFALMMAFLLLYVA

SEQ ID NO: 26 - DNA, 1493 bp, Arabidopsis thaliana
ggcttgacatgacaaatgtacaactgggagagaaagtcagtccgattgtgttggggatgacgatggcaaaagtagtaaataaggaagaaacaggaggggcgttttcgggagaagaaggaggaatatgagtgtgagttgttgttgtaggaatctgggcaagacaataaaaaaggcaataccttcacatcatttgcatctgagaagtcttggtgggagtctctatcgtcgtcgtatccaaagctcttcaatggagaccgatctcaagtcaacctttctcaacgtttattctgttctcaagtctgaccttcttcatgacccttccttcgaattcaccaatgaatctcgtctctgggttgatcggatgctggactacaatgtacgtggagggaaactcaatcggggtctctctgttgttgacagtttcaaacttttgaagcaaggcaatgatttgactgagcaagaggttttcctctcttgtgctctcggttggtgcattgaatggctccaagcttatttccttgtgcttgatgatattatggataactctgtcactcgccgtggtcaaccttgctggttcagagttcctcaggttggtatggttgccatcaatgatgggattctacttcgcaatcacatccacaggattctcaaaaagcatttccgtgataagccttactatgttgaccttgttgatttgtttaatgaggttgagttgcaaacagcttgtggccagatgatagatttgatcaccacctttgaaggagaaaaggatttggccaagtactcattgtcaatccaccgtcgtattgtccagtacaaaacggcttattactcattttatctccctgttgcttgtgcgttgcttatggccggcgaaaatttggaaaaccatattgatgtgaagaatgttcttgttgacatgggaatctacttccaagtgcaggatgattatctggattgttttgctgatcccgagacgcttggcaagataggaacagatatagaagatttcaaatgctcgtggttggtggttaaggcattagagcgctgcagcgaagaacaaactaagatattatatgagaactatggtaaacccgacccatcgaacgttgctaaagtgaaggatctctacaaagagctggatcttgagggagttttcatggagtatgagagcaaaagctacgagaagctgactggagcgattgagggacaccaaagtaaagcaatccaagcagtgctaaaatccttcttggctaagatctacaagaggcagaagtagtagagacagacaaacataagtctcagccctcaaaaatttcctgttatgtctttgattcttggttggtgatttgtgtaattctgttaagtgctctgattttcagggggaataataaacctgcctcacttttattcttgtgttacaattgtatttgtttcatgactatgatcttcttctttcatcagttatatgaatttgagattcttgttggttg

SEQ ID NO: 27 - protein, 384 aa, Arabidopsis thaliana
MSVSCCCRNLGKTIKKAIPSHHLHLRSLGGSLYRRRIQSSSMETDLKSTFLNVYSVLKSDLLHDPSFEFTNESRLWVDRMLDYNVRGGKLNRGLSVVDSFKLLKQGNDLTEQEVFLSCALGWCIEWLQAYFLVLDDIMDNSVTRRGQPCWFRVPQVGMVAINDGILLRNHIHRILKKHFRDKPYYVDLVDLFNEVELQTACGQMIDLITTFEGEKDLAKYSLSIHRRIVQYKTAYYSFYLPVACALLMAGENLENHIDVKNVLVDMGIYFQVQDDYLDCFADPETLGKIGTDIEDFKCSWLVVKALERCSEEQTKILYENYGKPDPSNVAKVKDLYKELDLEGVFMEYESKSYEKLTGAIEGHQSKAIQAVLKSFLAKIYKRQK

SEQ ID NO: 28 - DNA, 1647 bp, Citrus sinensis
atgtcgtctggagaaacatttcgtcctactgcagatttccatcctagtttatggagaaaccatttcctcaaaggtgcttctgatttcaagacagttgatcatactgcaactcaagaacgacacgaggcactgaaagaagaggtaaggagaatgataacagatgctgaagataagcctgttcagaagttacgcttgattgatgaagtacaacgcctgggggtggcttatcactttgagaaagaaataggagatgcaatacaaaaattatgtccaatctatattgacagtaatagagctgatctccacaccgtttcccttcattttcggttgcttaggcagcaaggaatcaagatttcatgtgatgtgtttgagaagttcaaagatgatgagggtagattcaagtcatcgttgataaacgatgttcaagggatgttaagtttgtacgaggcagcatacatggcagttcgcggagaacatatattagatgaagccattgctttcactaccactcacctgaagtcattggtagctcaggatcatgtaacccctaagcttgcggaacagataaatcatgctttataccgtcctcttcgtaaaaccctaccaagattagaggcgaggtattttatgtccatgatcaattcaacaagtgatcatttatgcaataaaactctgctgaattttgcaaagttagattttaacatattgctagagctgcacaaggaggaactcaatgaattaacaaagtggtggaaagatttagacttcactacaaaactaccttatgcaagagacagattagtggagttatatttttgggatttagggacatacttcgagcctcaatatgcatttgggagaaagataatgacccaattaaattacatattatccatcatagatgatacttatgatgcgtatggtacacttgaagaactcagcctctttactgaagcagttcaaagatggaatattgaggccgtagatatgcttccagaatacatgaaattgatttacaggacactcttagatgcttttaatgaaattgaggaagatatggccaagcaaggaagatcacactgcgtacgttatgcaaaagaggagaatcaaaaagtaattggagcatactctgttcaagccaaatggttcagtgaaggttacgttccaacaattgaggagtatatgcctattgcactaacaagttgtgcttacacattcgtcataacaaattccttccttggcatgggtgattttgcaactaaagaggtttttgaatggatctccaataaccctaaggttgtaaaagcagcatcagttatctgcagactcatggatgacatgcaaggtcatgagtttgagcagaagagaggacatgttgcgtcagctattgaatgttacacgaagcagcatggtgtctctaaggaagaggcaattaaaatgtttgaagaagaagttgcaaatgcatggaaagatattaacgaggagttgatgatgaagccaaccgtcgttgcccgaccactgctcgggacgattcttaatcttgctcgtgcaattgattttatttacaaagaggacgacggctatacgcattcttacctaattaaagatcaaattgcttctgtgctaggagaccacgttccattttga

SEQ ID NO: 29 - protein, 548 aa, Citrus sinensis
MSSGETFRPTADFHPSLWRNHFLKGASDFKTVDHTATQERHEALKEEVRRMITDAEDKPVQKLRLIDEVQRLGVAYHFEKEIGDAIQKLCPIYIDSNRADLHTVSLHFRLLRQQGIKISCDVFEKFKDDEGRFKSSLINDVQGMLSLYEAAYMAVRGEHILDEAIAFTTTHLKSLVAQDHVTPKLAEQINHALYRPLRKTLPRLEARYFMSMINSTSDHLCNKTLLNFAKLDFNILLELHKEELNELTKWWKDLDFTTKLPYARDRLVELYFWDLGTYFEPQYAFGRKIMTQLNYILSIIDDTYDAYGTLEELSLFTEAVQRWNIEAVDMLPEYMKLIYRTLLDAFNEIEEDMAKQGRSHCVRYAKEENQKVIGAYSVQAKWFSEGYVPTIEEYMPIALTSCAYTFVITNSFLGMGDFATKEVFEWISNNPKVVKAASVICRLMDDMQGHEFEQKRGHVASAIECYTKQHGVSKEEAIKMFEEEVANAWKDINEELMMKPTVVARPLLGTILNLARAIDFIYKEDDGYTHSYLIKDQIASVLGDHVPF

SEQ ID NO: 30 - DNA, 1641 bp, Artemisia annua
atgtcacttacagaagaaaaacctattcgccccattgccaactttcctccaagcatttggggagatcagtttctcatctatgaaaagcaagtagagcaaggggtggaacagatagtgaatgatttaaaaaaagaagtgcggcaactactaaaagaagctttggatattcctatgaaacatgccaatttgttgaagctgattgatgaaatccaacgccttggaataccgtatcactttgaacgggagattgatcatgcattgcaatgtatttatgaaacatatggtgataactggaatggtgaccgctcttccttatggttccgtcttatgcgaaagcaaggatattatgttacatgtgatgttttcaataactataaagacaaaaatggagcgttcaagcaatcgttagctaatgatgttgaaggtttgcttgagttgtacgaagcaacttctatgagggtacctggggagattatattagaagatgctcttggttttacacgatctcgtcttagcattatgacaaaagatgctttttctacaaaccccgctctttttaccgaaatacaacgggcactaaagcaacccctttggaaaaggttgccaagaatagaggcggcgcagtacattcctttctatcaacaacaagattctcataacaagactttacttaaacttgctaagttagagttcaatttgcttcagtcattgcacaaggaagagctcagccatgtgtgcaaatggtggaaagctttcgatatcaagaagaacgcaccttgtttaagagatagaattgttgaatgctacttttggggactaggttcaggctatgagccacagtattcccgggctagagttttcttcacaaaagctgttgctgttataactcttatagatgacacttatgatgcgtatggtacttatgaagaacttaagatctttactgaagctgttgaaaggtggtcaattacatgcttagacacacttccagaatacatgaaaccgatatacaaattattcatggatacatacacagaaatggaagaatttcttgcaaaggagggaagaacagatctatttaactgcggcaaagaatttgtgaaagagtttgttagaaacctgatgtttgaagcaaaatgggcaaatgagggacacataccaaccactgaagagcatgatccagttgtaatcattactggcggtgctaacctgcttacaacaacttgttatcttggcatgagtgatatattcacaaaagagtctgtcgaatgggctgtctctgcacctcctctttttagatactcaggtatacttggtcgacgcctaaatgatctcatgacccacaaggccgagcaagaaagaaaacatagttcatcgagccttgaaagttatatgaaggaatataatgtcaatgaggagtatgcccaaaccttgatttacaaggaagtagaagatgtgtggaaagatataaaccgagagtacctcacaactaaaaacattccaaggccgttattgatggctgtgatctatttgtgccagtttcttgaagttcaatatgcaggaaaggataacttcacacgtatgggagacgaatacaaacatctcataaagtctctactcgtttatcctatgagtatatga

SEQ ID NO: 31 - protein, 546 aa, Artemisia annua
MSLTEEKPIRPIANFPPSIWGDQFLIYEKQVEQGVEQIVNDLKKEVRQLLKEALDIPMKHANLLKLIDEIQRLGIPYHFEREIDHALQCIYETYGDNWNGDRSSLWFRLMRKQGYYVTCDVFNNYKDKNGAFKQSLANDVEGLLELYEATSMRVPGEIILEDALGFTRSRLSIMTKDAFSTNPALFTEIQRALKQPLWKRLPRIEAAQYIPFYQQQDSHNKTLLKLAKLEFNLLQSLHKEELSHVCKWWKAFDIKKNAPCLRDRIVECYFWGLGSGYEPQYSRARVFFTKAVAVITLIDDTYDAYGTYEELKIFTEAVERWSITCLDTLPEYMKPIYKLFMDTYTEMEEFLAKEGRTDLFNCGKEFVKEFVRNLMFEAKWANEGHIPTTEEHDPVVIITGGANLLTTTCYLGMSDIFTKESVEWAVSAPPLFRYSGILGRRLNDLMTHKAEQERKHSSSSLESYMKEYNVNEEYAQTLIYKEVEDVWKDINREYLTTKNIPRPLLMAVIYLCQFLEVQYAGKDNFTRMGDEYKHLIKSLLVYPMSI

SEQ ID NO: 32 - DNA, 1578 bp, Artificial sequence
  Yeast hydroxymethylglutaryl CoA reductase (HMG-R) the catalytic domain of HMG1 (tHMG)
atggaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 33 - protein, 525 aa, Artificial sequence
  Yeast hydroxymethylglutaryl CoA reductase (HMG-R) the catalytic domain of HMG1 (tHMG)
MDQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 34 - DNA, 1641 bp, Artemisia annua
atggccttgactgaagagaaacctataaggccaattgcaaatttcccaccttctatttggggcgatcaatttttgatttatgagaaacaagttgaacagggtgtggagcaaatagtaaacgatctaaagaaggaagtaagacagttgttaaaggaagcattggatattcctatgaaacatgcaaatttgttgaagctgattgacgagattcaacgtttaggtattccgtatcattttgaacgtgaaattgatcatgcattgcaatgtatttacgagacctatggtgataattggaatggcgacaggtctagcttatggtttaggctgatgcgtaaacaaggatactatgtcacgtgtgatgtgtttaataactataaagacaagaatggtgcttttaaacaatcgttagcgaatgatgttgaaggattgttggaattatatgaggctacgtccatgagagttccgggcgaaataattcttgaagatgccctgggattcacaagatcaaggctatcgattatgacaaaggacgcgtttagtacaaaccccgctttattcactgaaatccagagagctttaaagcaaccattgtggaagagattgccaaggatcgaggccgcccagtacatacccttctatcagcaacaagactcccataataaaactctgctaaagttagctaaactggagttcaatctattgcagagcctacataaggaagagttgagtcacgtatgcaagtggtggaaggcatttgatattaagaaaaatgccccatgtctgagagatcgtatcgttgaatgctatttttggggtttaggttctggttacgaaccccagtattcaagagctagagtttttttcactaaggcagttgctgtgataacacttattgatgacacgtatgacgcatatggaacttacgaggagttaaagattttcactgaagccgtggaaagatggtcgatcacctgcttagacacgttgccggaatacatgaagcctatttacaaattattcatggatacctacacagaaatggaggagtttctggccaaagaaggtagaactgatttattcaactgtgggaaggagttcgttaaagaattcgtaaggaatctaatggtagaagcgaaatgggctaatgaaggacacataccgacaactgaagagcacgatcccgtagttattatcactggtggtgcaaacttgctaacaactacctgttatctaggaatgagcgatattttcactaaggaatcagttgagtgggctgtatctgctccgcctttattccgttacagtggtattctgggtaggagattaaatgacctgatgactcataaagcggaacaggagagaaaacatagttcaagctctttagaatcttacatgaaggaatacaatgtcaacgaagaatatgctcagacactgatttataaggaagtggaggacgtttggaaagacataaacagagagtatttaacaacaaagaatatcccaagacctttacttatggctgttatatatctttgccaatttttagaggtccaatatgcaggtaaagacaattttacgagaatgggagatgaatacaagcatttgatcaaatcattgctagtttaccctatgtctatctaa

SEQ ID NO: 35 - protein, 546 aa, Artemisia annua
MALTEEKPIRPIANFPPSIWGDQFLIYEKQVEQGVEQIVNDLKKEVRQLLKEALDIPMKHANLLKLIDEIQRLGIPYHFEREIDHALQCIYETYGDNWNGDRSSLWFRLMRKQGYYVTCDVFNNYKDKNGAFKQSLANDVEGLLELYEATSMRVPGEIILEDALGFTRSRLSIMTKDAFSTNPALFTEIQRALKQPLWKRLPRIEAAQYIPFYQQQDSHNKTLLKLAKLEFNLLQSLHKEELSHVCKWWKAFDIKKNAPCLRDRIVECYFWGLGSGYEPQYSRARVFFTKAVAVITLIDDTYDAYGTYEELKIFTEAVERWSITCLDTLPEYMKPIYKLFMDTYTEMEEFLAKEGRTDLFNCGKEFVKEFVRNLMVEAKWANEGHIPTTEEHDPVVIITGGANLLTTTCYLGMSDIFTKESVEWAVSAPPLFRYSGILGRRLNDLMTHKAEQERKHSSSSLESYMKEYNVNEEYAQTLIYKEVEDVWKDINREYLTTKNIPRPLLMAVIYLCQFLEVQYAGKDNFTRMGDEYKHLIKSLLVYPMSI

SEQ ID NO: 36 - DNA, 1650 bp, Artemisia annua
ttgaaaatcatgtcacttacagaagaaaaacctattcgccccattgccaactttcctccaagcatttggggagatcagtttctcatctatgaaaagcaagtagagcaaggggtggaacagatagtgaatgatttaaaaaaagaagtgcggcaactactaaaagaagctttggatattcctatgaaacatgccaatttgttgaagctgattgatgaaatccaacgccttggaataccgtatcactttgaacgggagattgatcatgcattgcaatgtatttatgaaacatatggtgataactggaatggtgaccgctcttccttatggttccgtcttatgcgaaagcaaggatattatgttacatgtgatgttttcaataactataaagacaaaaatggagcgttcaagcaatcgttagctaatgatgttgaaggtttgcttgagttgtacgaagcaacttctatgagggtacctggggagattatattagaagatgctcttggttttacacgatctcgtcttagcattatgacaaaagatgctttttctacaaaccccgctctttttaccgaaatacaacgggcactaaagcaacccctttggaaaaggttgccaagaatagaggcggcgcagtacattcctttctatcaacaacaagattctcataacaagactttacttaaacttgctaagttagagttcaatttgcttcagtcattgcacaaggaagagctcagccatgtgtgcaaatggtggaaagctttcgatatcaagaagaacgcaccttgtttaagagatagaattgttgaatgctacttttggggactaggttcaggctatgagccacagtattcccgggctagagttttcttcacaaaagctgttgctgttataactcttatagatgacacttatgatgcgtatggtacttatgaagaacttaagatctttactgaagctgttgaaaggtggtcaattacatgcttagacacacttccagaatacatgaaaccgatatacaaattattcatggatacatacacagaaatggaagaatttcttgcaaaggagggaagaacagatctatttaactgcggcaaagaatttgtgaaagagtttgttagaaacctgatgtttgaagcaaaatgggcaaatgagggacacataccaaccactgaagagcatgatccagttgtaatcattactggcggtgctaacctgcttacaacaacttgttatcttggcatgagtgatatattcacaaaagagtctgtcgaatgggctgtctctgcacctcctctttttagatactcaggtatacttggtcgacgcctaaatgatctcatgacccacaaggccgagcaagaaagaaaacatagttcatcgagccttgaaagttatatgaaggaatataatgtcaatgaggagtatgcccaaaccttgatttacaaggaagtagaagatgtgtggaaagatataaaccgagagtacctcacaactaaaaacattccaaggccgttattgatggctgtgatctatttgtgccagtttcttgaagttcaatatgcaggaaaggataacttcacacgtatgggagacgaatacaaacatctcataaagtctctactcgtttatcctatgagtatataa

SEQ ID NO: 37 - protein, 546 aa, Artemisia annua; identical to SEQ ID NO: 31

SEQ ID NO: 38 - protein, 560 aa, Nicotiana plumbaginifolia
MASRRLLASLLRQSAQRGGGLISRSLGNSIPKSASRASSRASPKGFLLNRAVQYATSAAAPASQPSTPPKSGSEPSGKITDEFTGAGSIGKVCQVIGAVVDVRFDEGLPPILTALEVLDNQIRLVLEVAQHLGENMVRTIAMDGTEGLVRGQRVLNTGSPITVPVGRATLGRIINVIGEAIDERGPITTDHFLPIHREAPAFVEQATEQQILVTGIKVVDLLAPYQRGGKIGLFGGAGVGKTVLIMELINNVAKAHGGFSVFAGVGERTREGNDLYREMIESGVIKLGEKQSESKCALVYGQMNEPPGARARVGLTGLTVAEHFRDAEGQDVLLFIDNIFRFTQANSEVSALLGRIPSAVGYQPTLATDLGGLQERITTTKKGSITSVQAIYVPADDLTDPAPATTFAHLDATTVLSRQISELGIYPAVDPLDSTSRMLSPHILGEDHYNTARGVQKVLQNYKNLQDIIAILGMDELSEDDKMTVARARKIQRFLSQPFHVAEVFTGAPGKYVDLKESINSFQGVLDGKYDDLSEQSFYMVGGIDEVIAKAEKIAKESAA

SEQ ID NO: 39 - DNA, 1683 bp, Nicotiana plumbaginifolia
atggcttctcggaggcttctcgcctctctcctccgtcaatcggctcaacgtggcggcggtctaatttcccgatcgttaggaaactccatccctaaatccgcttcacgcgcctcttcacgcgcatcccctaagggattcctcttaaaccgcgccgtacagtacgctacctccgcagcggcaccggcatctcagccatcaacaccaccaaagtccggcagtgaaccgtccggaaaaattaccgatgagttcaccggcgctggttcgatcgggaaggtgtgccaggtcatcggtgccgtcgtggatgtgagattcgatgaaggtttgcccccaattttgaccgctctcgaagtgttggataatcagatccggcttgtgcttgaagtggctcagcatttgggcgagaatatggttaggactatagctatggatggtaccgaaggacttgttcgtggtcaacgcgtcctcaatactggttctcctatcaccgttcctgtcggtagagccacacttggccgtatcatcaatgtcattggagaggcaattgatgagagaggcccaattactaccgatcactttttgccaattcatcgtgaagctcctgcctttgtcgagcaagccactgaacaacaaattcttgtcactggtattaaggttgttgatcttctagctccataccaaagaggaggaaaaattgggctttttggtggtgctggtgtggggaaaactgtgcttattatggaactgattaacaatgttgcaaaagctcatggtggtttctctgtctttgctggtgttggtgaacgcactcgagagggtaatgatttgtaccgagaaatgattgaaagtggtgtcatcaagctaggcgagaagcaaagtgaaagcaagtgtgctcttgtatatggtcaaatgaatgagccccctggtgctcgtgcacgtgttggacttacaggtttgaccgtggctgagcacttccgagatgccgaggggcaggatgtgcttctctttattgacaatattttcaggtttactcaggctaactcagaagtgtctgctttgcttggtcgtatcccatctgctgtcggttatcaaccaactttggctacggatcttggaggtcttcaagaacgtatcaccaccaccaagaaaggttctattacatccgtgcaagctatttatgtgcctgctgatgacttgacagatccagcccctgctacaacctttgctcacttggatgccacaactgtcttgtctcgtcagatctctgagcttggtatctatcctgctgtcgatccacttgattctacatcccgtatgctctcgcctcacattttgggagaggatcactacaatactgctcgtggggtacagaaagttcttcaaaactacaagaatcttcaagatattattgctattttgggtatggatgagctcagtgaagatgataagatgacagttgcgcgtgcacgtaaaatccaaaggttccttagccagcctttccatgttgctgaagttttcacgggtgcccctggaaagtatgtcgacttgaaggagagcattaacagtttccagggagtgttggatggcaaatatgatgacctttcagagcaatcgttttatatggttggtggaatcgacgaggtcattgccaaagcagagaagattgccaaggaatctgctgcctag

SEQ ID NO: 40 - DNA, 741 bp, Arabidopsis thaliana
atggatatgcagaatgaaaacgagagattgatggtcttcgaacatgctcgcaaagtagcagaagcaacctacgtcaaaaaccctttagatgccgagaatttgacgagatgggcaggagctttacttgaactatcacagtttcagacagagccaaagcagatgattctagaggctattttgaagctgggagaggccttggtcatcgatccaaagaagcatgatgctctttggttaattgggaatgctcatctttcatttgggtttttgagttctgatcagacagaagctagcgataactttgagaaagcttctcagttctttcaacttgctgtggaggagcaaccagagagcgaactctatcggaaatcattgacattggcttccaaggctccagaactacatacaggcggcaccgctggaccatcatctaacagtgcgaagacgatgaagcagaaaaagaccagtgagttcaagtatgatgtgttcggatgggtcatcttagccagttacgttgttgcgtggatcagttttgccaattctcagacgccggtgtcaaggcagtaacgctcaccaagagcgtctaaagaccagcactttttttttaatgcattttgaaacaaaagagttttctcattataacaactcctagaacatatttgaagttaagaatgagagagactcagaaaatgtataagaactcatattgtcctttggaaattaacatacaaattagtgaagaca

SEQ ID NO: 41 - protein, 187 aa, Arabidopsis thaliana
MDMQNENERLMVFEHARKVAEATYVKNPLDAENLTRWAGALLELSQFQTEPKQMILEAILKLGEALVIDPKKHDALWLIGNAHLSFGFLSSDQTEASDNFEKASQFFQLAVEEQPESELYRKSLTLASKAPELHTGGTAGPSSNSAKTMKQKKTSEFKYDVFGWVILASYVVAWISFANSQTPVSRQ

SEQ ID NO: 42 - protein, 1017 aa, Arabidopsis thaliana
MVWFRAGSSVTKLAVRRILNQGASYATRTRSIPSQTRSFHSTICRPKAQSAPVPRAVPLSKLTDSFLDGTSSVYLEELQRAWEADPNSVDESWDNFFRNFVGQAATSPGISGQTIQESMRLLLLVRAYQVNGHMKAKLDPLGLEQREIPEDLDLALYGFTEADLDREFFLGVWQMSGFMSENRPVQTLRSILTRLEQAYCGNIGFEYMHIADRDKCNWLREKIETPTPWRYNRERREVILDRLAWSTQFENFLATKWTTAKRFGLEGGESLIPGMKEMFDRAADLGVESIVIGMSHRGRLNVLSNVVRKPLRQIFSEFSGGIRPVDEVGYTGTGDVKYHLGTSYDRPTRGGKKIHLSLVANPSHLEAADSVVVGKTRAKQYYSNDLDRTKNLGILIHGDGSFAGQGVVYETLHLSALPNYTTGGTIHIVVNNQVVFTTDPRAGRSSQYCTDVAKALSAPIFHVNGDDVEAVVHACELAAEWRQTFHSDVVVDLVCYRRFGHNEIDEPSFTQPKMYKVIKNHPSTLQIYHKKLLECGEVSQQDIDRIQEKVNTILNEEFVASKDYLPKKRDWLSTNWAGFKSPEQISRVRNTGVKPEILKTVGKAISSLPENFKPHRAVKKVYEQRAQMIESGEGVDWALAEALAFATLVVEGNHVRLSGQDVERGTFSHRHSVLHDQETGEEYCPLDHLIMNQDPEMFTVSNSSLSEFGVLGFELGYSMESPNSLVLWEAQFGDFANGAQVIFDQFISSGEAKWLRQTGLVMLLPHGYDGQGPEHSSARLERYLQMSDDNPYVIPDMEPTMRKQIQECNWQIVNATTPANYFHVLRRQIHRDFRKPLIVMAPKNLLRHKDCKSNLSEFDDVQGHPGFDKQGTRFKRLIKDQNDHSDLEEGIRRLVLCSGKVYYELDDERKKVGATDVAICRVEQLCPFPYDLIQRELKRYPNAEIVWCQEEAMNMGAFSYISPRLWTAMRSVNRGDMEDIKYVGRGPSAATATGFYTFHVKEQAGLVQKAIGKEPIN

SEQ ID NO: 43 - DNA, 3054 bp, Arabidopsis thaliana
atggtgtggtttcgtgctggttccagtgttacaaagctagctgttagaaggattttgaatcagggtgcttcgtatgcgacgaggacacggtctattccgtctcaaactcgttcctttcactcgactatatgcagaccaaaggctcagagtgctccagttcctagagctgttcctctttctaagctaactgatagtttcttagatgggacgagcagtgtctaccttgaggagttacaaagggcttgggaagctgatcctaacagtgtagatgagtcttgggataatttctttaggaactttgttggtcaggctgccacgtctcctggcatctctgggcagacaattcaggagagtatgaggctgttattacttgttagggcttatcaggtgaatggtcacatgaaagcgaagttggatccgttaggtttggaacagcgagagatccctgaggatcttgacttggctctttatggattcactgaggctgaccttgacagagagttcttcttgggggtgtggcagatgtcaggattcatgtctgagaaccgaccagtgcagacccttcgttccatattgacaaggctcgaacaggcatactgtgggaatatcggatttgagtatatgcacattgcagatcgagataaatgtaactggttgagagaaaagattgagacaccaactccttggcggtacaacagggagcgccgtgaggtgattctcgatcggcttgcatggagtactcagttcgagaatttcttagctaccaagtggacaacagccaaaagatttggacttgagggaggagaatcattaattcctggaatgaaggagatgtttgacagagcagcagatcttggagtagagagtattgttattggaatgtctcacagaggaagattgaatgttctgagtaatgttgttcggaagccactccgtcagatatttagtgagttcagtggtggtattaggcctgtagatgaagttggctacactggaactggtgatgtcaaatatcacttgggaacctcttatgatcgacctacaagaggtgggaagaaaatccatctctctttggttgctaatccaagtcacttggaagctgcagattctgttgttgttggcaaaaccagagcaaaacagtactactccaatgatttagacaggaccaaaaatttaggtattttgattcacggagatggtagttttgctggacaaggggtagtctatgaaactctccatcttagtgctcttccaaactacaccaccggaggaaccatacatattgtggtgaacaaccaagtggttttcacgacagatccaagggcggggagatcttcccagtattgtactgatgttgcaaaggctttgagtgctcccatctttcatgttaatggggatgatgttgaggctgttgttcatgcctgcgagcttgctgctgagtggcgtcagacttttcattctgatgttgtcgttgatttggtttgctaccgtaggttcgggcataatgagatagatgaaccatctttcactcagccaaaaatgtacaaggttatcaaaaatcatccttcaacccttcagatctaccacaaaaagctcttggaatgcggtgaagtatcacaacaggatattgaccggatacaggaaaaggttaacaccatcctcaatgaagaatttgtcgctagtaaggactatctccctaagaaacgagattggctttcaaccaattgggctggatttaagtctcctgagcagatctcacgtgttagaaacactggcgtcaaaccagagatactgaagactgttggcaaggcaatttcatctcttccagaaaacttcaagccacacagggcagtgaagaaagtttatgaacaacgtgcccaaatgattgaatcaggagagggagttgactgggcccttgcagaagctcttgcttttgctaccttagttgtggaaggcaatcatgtccgattgagtggtcaggatgtcgaacgaggaacatttagtcatcgtcattctgtccttcatgaccaggaaactggagaagagtattgtcctctagatcatctcatcatgaatcaggatcctgagatgtttactgttagcaacagttctctttcagaatttggtgtccttgggttcgaattgggttactccatggaaagcccgaactcgttggtactatgggaagctcagtttggagacttcgccaatggagctcaggtgatatttgatcagttcatcagcagtggagaagccaaatggctgcgtcaaaccgggcttgttatgctacttccccatggttatgatggtcagggacctgaacattcaagtgcgaggttggaacgttaccttcagatgagtgatgataatccctatgtcataccagacatggaaccaacaatgcgaaagcaaattcaagaatgtaattggcagattgtcaatgccacaactcccgccaactatttccatgttctgcggcgacagatacacagagacttccgtaagcctctgattgtaatggcaccaaagaacttgctccgtcacaaggactgcaaatcaaatctctcagagtttgatgatgtccaaggccacccaggttttgacaagcaaggaactagatttaagcgattaatcaaggatcagaatgatcactctgatcttgaagaaggcatcagaagattggtactttgctccggaaaggtctattatgagcttgatgatgaacggaagaaggttggcgcaacagatgttgctatctgtagagttgaacagctttgtcctttcccatatgatctcattcagcgtgagctcaagagatatccaaatgcggagatcgtttggtgccaagaagaggcgatgaacatgggagcattcagctacatatctccacggctatggacagcaatgagaagcgtaaacagaggagatatggaagacattaagtatgttggtcgtggtccttctgctgcaactgccacgggtttctatactttccatgtcaaagagcaagccgggcttgtccagaaagccatcggaaaggaacccatcaattaa

SEQ ID NO: 44 - DNA, 87 bp, Saccharomyces cerevisiae
atgctttcactacgtcaatctataagatttttcaagccagccacaagaactttgtgtagctctagatatctgcttcagcaaaaaccc

SEQ ID NO: 45 - protein, 29 aa, Saccharomyces cerevisiae
MLSLRQSIRFFKPATRTLCSSRYLLQQKP

SEQ ID NO: 46 - DNA, 66 bp, Saccharomyces cerevisiae
atgttgagatcatccgttgttcgtagtcgcgctactttaaggcctttattgcgtcgtgcttactcc

SEQ ID NO: 47 - protein, 22 aa, Saccharomyces cerevisiae
MLRSSVVRSRATLRPLLRRAYS

SEQ ID NO: 48 - DNA, 69 bp, Saccharomyces cerevisiae
atgcttgctgctaaaaacatactaaacaggtcaagcttgtctagctctttccgtattgccacacgtttg

SEQ ID NO: 49 - protein, 23 aa, Saccharomyces cerevisiae
MLAAKNILNRSSLSSSFRIATRL

SEQ ID NO: 50 - DNA, 93 bp, Saccharomyces cerevisiae
atgctaaaatacaaacctttactaaaaatctcgaagaactgtgaggctgctatcctcagagcgtctaagactagattgaacacaatccgcgcg

SEQ ID NO: 51 - protein, 31 aa, Saccharomyces cerevisiae
MLKYKPLLKISKNCEAAILRASKTRLNTIRA

SEQ ID NO: 52 - DNA, 225 bp, Artificial sequence
  Presequence of the subunit 9 of the F0 ATPase of Neurospora crassa  coding sequence
atgatacaagtagctaaaataataggaacagggctagctaccacaggtttaatcggagctggtataggtattggagttgtatttggctcattaataataggggtttcaagaaacccttcgttaaaaagtcaattatttgcatatgcaattttaggttttgctttctcggaagcgacaggattatttgctttgatgatggcttttttacttctttatgttgcatag